(19) United States
(12) Patent Application Publication
(10) Pub. No.: US 2026/0131061 A1
(43) Pub. Date: May 14, 2026

(54) ON-SITE PRODUCTION OF DIALYSATE SOLUTION AND VARIABLE CONCENTRATION DOSING

(71) Applicant: Fresenius Medical Care Holdings, Inc., Waltham, MA (US)

(72) Inventors: Kulwinder Plahey, Martinez, CA (US); Karsten Fischer, Nashua, NH (US); Connor Cassis, Natick, MA (US); Kendall Cox, Swampscott, MA (US)

(21) Appl. No.: 18/948,083

(22) Filed: Nov. 14, 2024

(51) Int. Cl. A61M 1/28; A61M 1/14

(52) U.S. Cl. CPC A61M 1/287; A61M 1/152; A61M 1/155; A61M 1/159; A61M 2202/0007; A61M 2202/0014; A61M 2202/04; A61M 2205/10; A61M 2205/121; A61M 2205/50

(57) ABSTRACT

On-site production of dialysate solution can be accomplished by mixing a base dialysate solution at a low concentration of dextrose with a concentrated dialysate solution at a high concentration of dextrose, allowing for weight savings when shipping dialysate solution to a patient. The base dialysate solution can be mixed in a large reservoir using purified water provided by a water purifier on-site, allowing for a small amount of dextrose or electrolytes to be mixed with the water to create tens of liters of dialysate solution. A peritoneal dialysis machine can be configured to mix the base dialysate solution from the reservoir with the concentrated dialysate solution to create, on-demand, a target dosage dialysate solution at any desired target concentration.

### BACKGROUND

Dialysis is a treatment used to support a patient with insufficient renal function. The two principal treatment options are hemodialysis (HD) and peritoneal dialysis (PD). During hemodialysis, the patient's blood is removed, e.g., via an arteriovenous (AV) fistula or other methods (e.g., AV graft), and passed through a dialyzer of a dialysis machine while also passing a dialysis solution, referred to as dialysate, through the dialyzer. A semi-permeable membrane in the dialyzer separates the blood from the dialysate within the dialyzer and facilitates the exchange of waste products (e.g., urea, creatine, potassium, etc.) between the blood stream and the dialysate. The membrane prevents the transfer of blood cells, protein, and other important components in the blood stream from entering the dialysate solution. The cleaned blood stream is then returned to the patient's body. In this way, the dialysis machine functions as an artificial kidney for cleaning the blood in patients with insufficient renal function.

In contrast with hemodialysis, the peritoneal dialysis treatment option introduces dialysate into a patient's peritoneal cavity, which is an area in the abdomen between the parietal peritoneum and visceral peritoneum (e.g., a space between the membrane that surrounds the abdominal wall and the membranes that surround the internal organs in the abdomen). The lining of the patient's peritoneum functions as a semi-permeable membrane that facilitates the exchange of waste product between the bloodstream and the dialysate, similar in function to the membrane in the dialyzer of the hemodialysis machine. The patient's peritoneal cavity is drained and filled with new dialysate over a number of PD cycles. Peritoneal dialysis can be performed using either gravity or an automated pumping mechanism to fill and drain the abdomen during a PD cycle.

Automated PD machines, sometimes referred to as PD cyclers, are designed to control the PD treatment process so that it can be performed at home without clinical staff, typically while the patient sleeps overnight so as to minimize interference with the patient's life. The process is referred to as continuous cycler-assisted peritoneal dialysis (CCPD). Many PD cyclers are designed to automatically infuse, dwell, and drain dialysate to and from the peritoneal cavity. The PD treatment typically lasts several hours, often beginning with an initial drain phase to empty the peritoneal cavity of used or spent dialysate that was left in the peritoneal cavity at the end of the last PD treatment. The sequence then proceeds through a progression of fill, dwell, and drain phases that follow sequentially. A group of fill, dwell, and drain phases, in order, can be referred to as a PD cycle.

Each PD treatment may consist of one or more PD cycles, and each cycle may commonly fill the patient's abdomen with 2-3 liters of clean dialysate solution. The dialysate solution utilized with a PD cycler is a sterile solution of water, dextrose or glucose, and a number of electrolytes (e.g., sodium, potassium, calcium, magnesium, chloride, and/or bicarbonate). The dialysate solution is usually provided in bags having a volume between 1.5 and 5 liters. Based on the required PD treatment, a number of dialysis bags may be connected to the PD cycler and used during each treatment. This presents a challenge when PD treatment is performed at the patient's residence or other location; namely, the dialysate bags needed to be shipped to the patient, and the weight and volume of bags necessary for treatment is not insignificant as each liter of solution weighs approximately 1 kg.

When considering how to reduce costs and improve the experience of patients undergoing PD treatment at home, the question of how to transport all of the necessary supplies to the location of treatment should be carefully considered. Thus, there is a desire to improve all aspects of PD treatment for the patient.

### SUMMARY

A dialysate production system can be used in combination with an existing PD machine to mix a desired target dosage concentration of dialysate solution on demand. A filtration and purification system can generate clean water from an available water source, and mix the clean water with a small amount of electrolyte and/or dextrose to generate a reservoir of base dialysate solution of specific concentration (e.g., 0-1.5% dextrose). A fluid line is attached from the reservoir to a disposable cassette of the PD machine. A second bag of concentrated dextrose solution (e.g., 50% dextrose) can be attached to a second fluid line of the disposable cassette. The PD machine can then mix the based dialysate solution with the concentrated dialysate solution in the pump chamber of the disposable cassette or the fluid lines (e.g., a patient line) to create the desired target concentration of dialysate solution. This allows for on-demand dosing of dialysate solution having any concentration between the base concentration and the high concentration.

In accordance with a first aspect of the disclosure, a system is provided for generating dialysate solution for peritoneal dialysis treatments. The system includes: a water purifier coupled to a water source; a reservoir to hold base dialysate solution at a first concentration, wherein the base dialysate solution includes a mixture of purified water from the water purifier and at least one of electrolytes or dextrose; and a PD machine that accepts a disposable cassette. The PD machine is configured to mix a first volume of the base dialysate solution from the reservoir with a second volume of concentrated dialysate solution at a second concentration to create a target dosage dialysate solution at a third concentration between the first concentration and the second concentration.

In some embodiments of the first aspect, the water purifier comprises at least one of a mechanical filter, an activated carbon filter, a reverse osmosis membrane, or a deionization filter.

In some embodiments of the first aspect, the water purifier further comprises a sterilization component that comprises at least one of a ultraviolet light source or an endotoxin filter.

In some embodiments of the first aspect, the first concentration of the base dialysate solution is between 0.0% and 1.5% by weight of dextrose.

In some embodiments of the first aspect, the second concentration of the base dialysate solution is between 4.5% and 50% by weight of dextrose.

In some embodiments of the first aspect, the first concentration of the base dialysate solution is 0.0% by weight of dextrose, and the base dialysate solution contains at least some concentration of electrolytes.

In some embodiments of the first aspect, the disposable cassette is connected to a first fluid line connected to the reservoir. The disposable cassette is also connected to a second fluid line connected to a dialysate bag containing the concentrated dialysate solution.

In some embodiments of the first aspect, the PD machine is configured to: configure the disposable cassette to create a first fluid path between the first fluid line and a first pump chamber; draw the first volume of the base dialysate solution from the reservoir into the first pump chamber; configure the disposable cassette to create a second fluid path between the second fluid line and the first pump chamber; draw the second volume of the concentrated dialysate solution from the dialysate bag containing the concentrated dialysate solution into the first pump chamber; configure the disposable cassette to create a third fluid path between the first pump chamber and a patient line; and pump the first volume of the base dialysate solution and the second volume of the concentrated dialysate solution into the patient line.

In some embodiments of the first aspect, the PD machine is configured to mix the first volume of the base dialysate solution and the second volume of the concentrated dialysate solution in the first pump chamber. In other embodiments of the first aspect, the PD machine is configured to mix the first volume of the base dialysate solution and the second volume of the concentrated dialysate solution, at least partially, in the patient line. In some embodiments of the first aspect, the PD machine is configured to mix the first volume of the base dialysate solution and the second volume of the concentrated dialysate solution in a mixing bag fluidly coupled with the disposable cassette via a third fluid line.

In some embodiments of the first aspect, the PD machine is configured to use partial strokes of a piston to draw at least one of the first volume of the base dialysate solution or the second volume of the concentrated dialysate solution into the pump chamber.

In some embodiments of the first aspect, the PD machine is further configured to pump a volume of the dosage dialysate solution into a patient line attached to the disposable cassette, wherein the patient line is connected to a catheter inserted into a peritoneal cavity of a patient.

In accordance with a second aspect of the present disclosure, a peritoneal dialysis machine is provided. The PD machine includes at least one pump mechanism proximate an interface for a disposable cassette, and at least one processor coupled to a memory. The memory stores instructions that, when executed by the at least one processor, cause the PD machine to: mix a first volume of a base dialysate solution at a first concentration from a reservoir with a second volume of concentrated dialysate solution at a second concentration to create a target dosage dialysate solution at a third concentration between the first concentration and the second concentration.

In some embodiments of the second aspect, the first concentration of the base dialysate solution is between 0.0% and 1.5% by weight of dextrose.

In some embodiments of the second aspect, the second concentration of the base dialysate solution is between 4.5% and 50% by weight of dextrose.

In some embodiments of the second aspect, the first concentration of the base dialysate solution is 0.0% by weight of dextrose, and the base dialysate solution contains at least some concentration of electrolytes.

In some embodiments of the second aspect, the disposable cassette is connected to a first fluid line connected to the reservoir, and wherein the disposable cassette is connected to a second fluid line connected to a dialysate bag containing the concentrated dialysate solution.

In some embodiments of the second aspect, the PD machine is configured to: configure the disposable cassette to create a first fluid path between the first fluid line and a first pump chamber; draw the first volume of the base dialysate solution from the reservoir into the first pump chamber; configure the disposable cassette to create a second fluid path between the second fluid line and the first pump chamber; draw the second volume of the concentrated dialysate solution from the dialysate bag containing the concentrated dialysate solution into the first pump chamber; configure the disposable cassette to create a third fluid path between the first pump chamber and a patient line; and pump the first volume of the base dialysate solution and the second volume of the concentrated dialysate solution into the patient line.

In some embodiments of the second aspect, the PD machine is configured to mix the first volume of the base dialysate solution and the second volume of the concentrated dialysate solution in the first pump chamber. In other embodiments of the second aspect, the PD machine is configured to mix the first volume of the base dialysate solution and the second volume of the concentrated dialysate solution, at least partially, in the patient line. In some embodiments of the second aspect, the PD machine is configured to mix the first volume of the base dialysate solution and the second volume of the concentrated dialysate solution in a mixing bag fluidly coupled with the disposable cassette via a third fluid line.

In accordance with a third aspect of the present disclosure, a method for operating a peritoneal dialysis (PD) machine is provided. The method includes: purifying water using a water purifier to generate purified water; mixing at least one of electrolytes or dextrose with the purified water in a reservoir to create a base dialysate solution at a first concentration; mixing, using a disposable cassette of the PD machine, a first volume of the base dialysate solution from the reservoir with a second volume of concentrated dialysate solution at a second concentration to create a target dosage dialysate solution at a third concentration between the first concentration and the second concentration.

In some embodiments of the third aspect, the disposable cassette is connected to a first fluid line connected to the reservoir. The disposable cassette is also connected to a second fluid line connected to a dialysate bag containing the concentrated dialysate solution.

In some embodiments of the third aspect, wherein the mixing the first volume of the base dialysate solution with the second volume of concentrated dialysate solution comprises: configuring the disposable cassette to create a first fluid path between the first fluid line and a first pump chamber; drawing the first volume of the base dialysate solution from the reservoir into the first pump chamber; configuring the disposable cassette to create a second fluid path between the second fluid line and the first pump chamber; drawing the second volume of the concentrated dialysate solution from the dialysate bag containing the concentrated dialysate solution into the first pump chamber; configuring the disposable cassette to create a third fluid path between the first pump chamber and a patient line; and pumping the first volume of the base dialysate solution and the second volume of the concentrated dialysate solution into the patient line.

In some embodiments of the third aspect, the PD machine is configured to mix the first volume of the base dialysate solution and the second volume of the concentrated dialysate solution in the first pump chamber. In other embodiments of the third aspect, the PD machine is configured to mix the first volume of the base dialysate solution and the second volume of the concentrated dialysate solution, at least partially, in the patient line. In some embodiments of the third aspect, the PD machine is configured to mix the first volume of the base dialysate solution and the second volume of the concentrated dialysate solution in a mixing bag fluidly coupled with the disposable cassette via a third fluid line.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1 illustrates a peritoneal dialysis (PD) system, in accordance with some embodiments.

FIG. 2 is a perspective view of the PD machine and a disposable cassette of the PD system of FIG. 1, in accordance with some embodiments

FIG. 3 is a perspective view of an open cassette compartment of the PD machine of FIG. 1, in accordance with some embodiments.

FIG. 4 is an exploded, perspective view of the disposable cassette of FIG. 2, in accordance with some embodiments.

FIG. 5 is a cross-sectional view of the fully assembled disposable cassette of FIG. 2, in accordance with some embodiments.

FIGS. 6A and 6B are perspective views of the disposable cassette of FIG. 2 from a front side and a back side, respectively, in accordance with some embodiments.

FIG. 6C illustrates the disposable cassette seated against the cassette interface, in accordance with some embodiments.

FIG. 7A is a perspective view of another embodiment of a peritoneal dialysis system.

FIGS. 7B and 7C are perspective and plan views of another embodiment of a cassette for use with the peritoneal dialysis system of FIG. 7A.

FIG. 7D is a schematic fluid flow diagram of the peritoneal dialysis system of FIG. 7A.

FIGS. 8A and 8B are perspective views of another embodiment of a peritoneal dialysis system, including a disposable dialysis cassette.

FIG. 8C illustrates an embodiment of a warming cassette for use with the peritoneal dialysis machine and disposable dialysis cassette of FIGS. 8A and 8B.

FIG. 9 illustrates a system for producing dialysate solution at the point of care, in accordance with some embodiments.

FIGS. 10A-10F are conceptual illustrations of the fluid pathways for accomplishing mixing of dialysate solution to a final target dosage concentration using the disposable cassette, in accordance with some embodiments.

FIG. 11 is a flow diagram of a method for operating a peritoneal dialysis machine, in accordance with some embodiments.

FIG. 12 is a flow diagram of a method for mixing two dialysate solutions using a first mixing modality, in accordance with some embodiments.

FIG. 13 is a flow diagram of a method for mixing two dialysate solutions in accordance with another mixing modality, in accordance with some embodiments.

FIG. 14 illustrates a schematic fluid flow diagram for a dialysis system having a peristaltic pump and utilizing a mixing bag, in accordance with some embodiments.

FIG. 15 illustrates an exemplary computer system, in accordance with some embodiments.

### DETAILED DESCRIPTION

A system for generating on-demand dialysate solution for use with a PD machine is described below. The system is intended for performing peritoneal dialysis at remote locations in contrast with a hemodialysis clinic, hospital, or other medical facility. To reduce the amount of material that must be shipped to a remote location, the present embodiments leverage the ability of dialysate solution to be mixed, on demand, anywhere there is a sufficient water supply, which advantageously reduces the overall weight and volume of dialysate that must be shipped to a patient because dextrose and electrolytes can be shipped in bags at much higher concentrations than intended for treatment, and then diluted on-site with a low-concentration base dialysate solution made on-site according to embodiments.

In various embodiments, a PD machine may be adapted to mix the two solutions using the pump mechanisms of the machines, where the solutions are mixed in the pump chambers of a disposable cassette and/or the fluid lines connected between the cassette and the patient. In other embodiments, a PD machine may be adapted to mix the two solutions using a mixing bag or container. Exemplary PD machines adapted for these uses are described in more detail below.

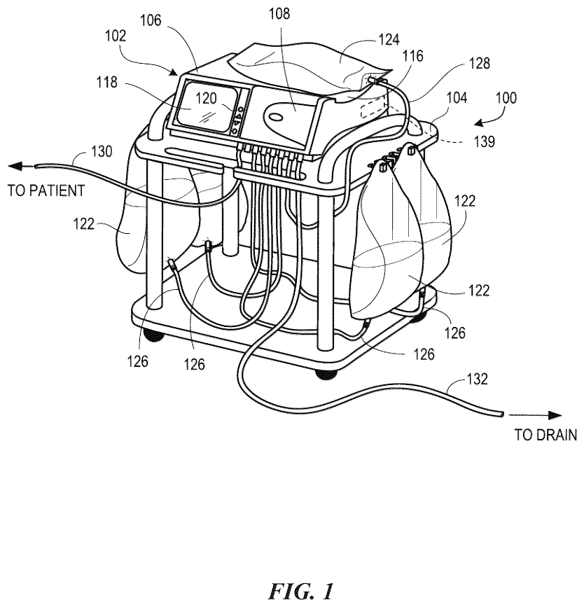

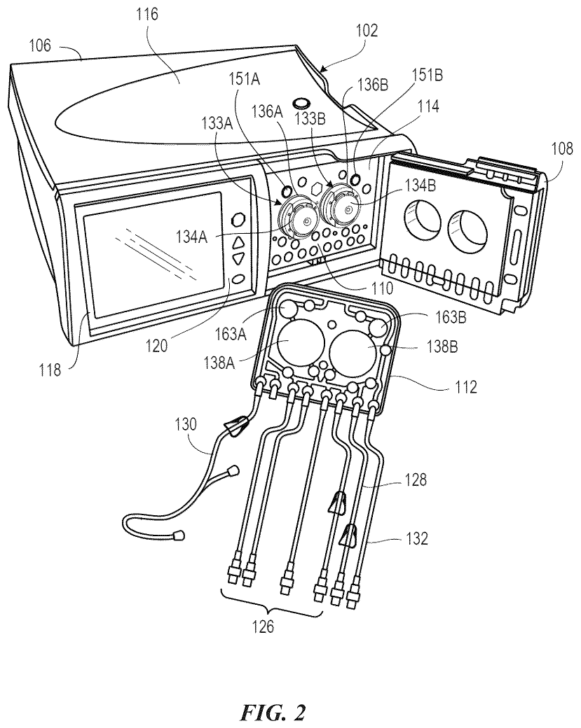

FIG. 1 illustrates a peritoneal dialysis (PD) system 100, in accordance with some embodiments. The PD system 100 can include a PD machine 102, which can alternately be referred to as a PD cycler, seated on a cart 104. The PD machine 102 includes a housing 106, a door 108, and a cassette interface 110 that contacts a disposable PD cassette 112 when the cassette 112 is disposed within a cassette compartment 114 formed between the cassette interface 110 and the closed door 108. The cassette compartment 114, cassette interface 110, and cassette 112 are shown in more detail in FIG. 2. A heater tray 116 is positioned on top of the housing 106. The heater tray 116 is sized and shaped to accommodate a bag of PD solution such as dialysate (e.g., a 5 liter bag of dialysate). The PD machine 102 also includes a user interface such as a touch screen display 118 and additional control buttons 120 that can be operated by a user (e.g., a caregiver or a patient) to allow, for example, set up, initiation, and/or termination of a PD treatment.

Dialysate bags 122 are suspended from fingers on the sides of the cart 104, and a heater bag 124 is positioned in the heater tray 116. The dialysate bags 122 and the heater bags 124 are connected to the cassette 112 via dialysate bag lines 126 and a heater bag line 128, respectively. The dialysate bag lines 126 can be used to pass dialysate from dialysate bags 122 to the cassette 112 during use, and the heater bag line 128 can be used to pass dialysate back and forth between the cassette 112 and the heater bag 124 during use. In addition, a patient line 130 and a drain line 132 are connected to the cassette 112. The patient line 130 can be connected to a patient's abdomen via a catheter and can be used to pass dialysate back and forth between the cassette 112 and the patient's peritoneal cavity during use. The catheter may be surgically implanted in the patient and connected to the patient line 130 via a port, such as a fitting, prior to the PD treatment. The drain line 132 can be connected to a drain or drain receptacle and can be used to pass dialysate from the cassette 112 to the drain or drain receptacle during use.

The PD machine 102 also includes a control unit 139 (e.g., a processor, controller, system-on-chip (SoC), or the like). The control unit 139 can receive signals from and transmit signals to the touch screen display 118, the control panel 120, and the various other components of the PD system 100. The control unit 139 can control the operating parameters of the PD machine 102. In some embodiments, the control unit 139 includes an MPc823 PowerPC device manufactured by Motorola, Inc. As further discussed in detail elsewhere herein, in some embodiments, the control unit 139 may be configured to control disengaging and/or bypassing of a pump in connection with naturally draining the dialysate from a patient during the drain phase of a PD cycle.

FIG. 2 is a perspective view of the PD machine 102 and the PD cassette 112 of the PD system 100 of FIG. 1, in accordance with some embodiments. As depicted in FIG. 2, the PD cassette 112 is placed proximate the cassette interface 110. The cassette 112 contains pump chambers 138A, 138B, pressure sensing chambers 163A, 163B, and valve chambers for controlling the flow of fluid through the cavities of the cassette 112. The cassette 112 is connected to the dialysate bag lines 126, the heater bag line 128, the patient line 130, and the drain line 132.

The cassette interface 110 includes a surface having holes formed therein. The PD machine 102 includes pistons 133A, 133B with piston heads 134A, 134B attached to piston shafts. The piston shafts can be actuated to move the piston heads 133A, 133B axially within piston access ports 136A, 136B formed in the cassette interface 110. The pistons 133A, 133B are sometimes referred to herein as pumps. In some embodiments, the piston shafts can be connected to stepper motors that can be operated to move the pistons 133A, 133B axially inward and outward such that the piston heads 134A, 134B move axially inward and outward within the piston access ports 136A, 136B. The stepper motors drive lead screws, which move nuts inward and outward on the lead screws. The stepper motors can be controlled by driver modules. The nuts, in turn, are connected to the piston shafts, which cause the piston heads 134A, 134B to move axially inward and outward as the stepper motors drive the lead screws. Stepper motor controllers provide the necessary current to be driven through the windings of the stepper motors to move the pistons 133A, 133B. The polarity of the current determines whether the pistons 133A, 133B are advanced or retracted. In some embodiments, the stepper motors require 200 steps to make a full rotation, and this corresponds to 0.048 inches of linear travel of the piston heads 134A, 134B.

In some embodiments, the PD system 100 also includes encoders (e.g., optical quadrature encoders) that measure the rotational movement and direction of the lead screws. The axial positions of the pistons 133A, 133B can be determined based on the rotational movement of the lead screws, as indicated by feedback signals from the encoders. Thus, measurements of the position calculated based on the feedback signals can be used to track the position of the piston heads 134A, 134B of the pistons 133A, 133B.

When the cassette 112 is positioned within the cassette compartment 114 of the PD machine 102 with the door 108 closed, the piston heads 134A, 134B of the PD machine 102 align with the pump chambers 138A, 138B of the cassette 112 such that the piston heads 134A, 134B can be mechanically connected to dome-shaped fastening members of the cassette 112 overlying the pump chambers 138A, 138B. As a result of this arrangement, movement of the piston heads 134A, 134B toward the cassette 112 during treatment can decrease the volume of the pump chambers 138A, 138B and force dialysate out of the pump chambers 138A, 138B. Retraction of the piston heads 134A, 134B away from the cassette 112 can increase the volume of the pump chambers 138A, 138B and cause dialysate to be drawn into the pump chambers 138A, 138B.

The cassette 112 also includes pressure sensor chambers 163A, 163B. When the cassette 112 is positioned within the cassette compartment 114 of the PD machine 102 with the door 108 closed, pressure sensors 151A, 151B align with the pressure sensor chambers 163A, 163B. Portions of a membrane that overlies the pressure sensor chambers 163A, 163B adhere to the pressure sensors 151A, 151B using vacuum pressure. Specifically, clearance around the pressure sensors 151A, 151B communicates vacuum to the portions of the cassette membrane overlying the pressure sensing chambers 163A, 163B to hold those portions of the cassette membrane tightly against the pressure sensors 151A, 151B. The pressure of fluid within the pressure sensing chambers 163A, 163B causes the portions of the cassette membrane overlying the pressure sensor chambers 163A, 163B to contact and apply a force to the pressure sensors 151A, 151B.

The pressure sensors 151A, 151B can be any sensors that are capable of measuring the fluid pressure in the pressure sensor chambers 163A, 163B. In some embodiments, the pressure sensors are solid state silicon diaphragm infusion pump force/pressure transducers. One example of such a sensor is the model 1865 force/pressure transducer manufactured by Sensym® Foxboro ICT. In some embodiments, the force/pressure transducer is modified to provide increased voltage output. The force/pressure transducer can, for example, be modified to produce an output signal of 0 to 5 volts.

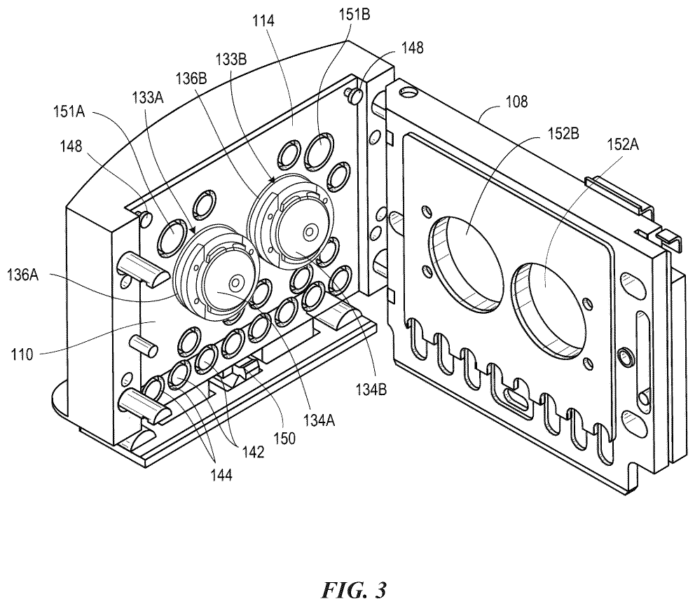

FIG. 3 is a perspective view of an open cassette compartment 114 of the PD machine 102 of FIG. 1, in accordance with some embodiments. As discussed above, the PD machine 102 includes pistons 133A, 133B disposed in piston access ports 136A, 136B, respectively. The PD machine 102 also includes multiple inflatable members 142 positioned within inflatable member ports 144 in the cassette interface 110. The inflatable members 142 align with depressible dome regions of the cassette 112 when the cassette 112 is positioned within the cassette compartment 114 of the PD machine 102. While only a couple of the inflatable members 142 are labeled in FIG. 3, it should be understood that the PD machine 102 includes an inflatable member 142 associated with each of the depressible dome regions of the cassette 112. The inflatable members 142 act, in cooperation with the depressible dome regions, as valves to direct dialysate through the cassette 112 in a desired manner during use. In particular, the inflatable members 142 bulge outward beyond the surface of the cassette interface 110 and into contact with the depressible dome regions of the cassette 112 when inflated, and retract into the inflatable member ports 144 and out of contact with the cassette 112 when deflated. By inflating certain inflatable members 142 to depress their associated dome regions on the cassette 112, certain fluid flow paths within the cassette 112 can be occluded. Thus, dialysate can be pumped through the cassette 112 by actuating the piston heads 134A, 134B, and can be guided along desired flow paths within the cassette 112 by selectively inflating and deflating the various inflatable members 142.

In some embodiments, locating pins 148 extend from the cassette interface 110 of the PD machine 102. When the door 108 is in the open position, the cassette 112 can be loaded onto the cassette interface 110 by positioning the top portion of the cassette 112 under the locating pins 148 and pushing the bottom portion of the cassette 112 toward the cassette interface 110. The cassette 112 is dimensioned to remain securely positioned between the locating pins 148 and a spring loaded latch 150 extending from the cassette interface 110 to allow the door 108 to be closed over the cassette 112. The locating pins 148 help to ensure that proper alignment of the cassette 112 within the cassette compartment 114 is maintained during use.

The door 108 of the PD machine 102 defines cylindrical recesses 152A, 152B that substantially align with the pistons 133A, 133B when the door 108 is in the closed position. When the cassette 112 is positioned within the cassette compartment 114 with the door 108 closed, the pump chambers 138A, 138B at least partially fit within the recesses 152A, 152B. The door 108 further includes a pad that is inflated during use to compress the cassette 112 between the door 108 and the cassette interface 110. With the pad inflated, the portions of the door 108 forming the recesses 152A, 152B support the surface of the pump chambers 138A, 138B, and the other portions of the door 108 support the other regions or surfaces of the cassette 112. The door 108 can counteract the forces applied by the inflatable members 142 and, therefore, allows the inflatable members 142 to actuate the depressible dome regions on the cassette 112. The engagement between the door 108 and the cassette 112 can also help to hold the cassette 112 in a desired position within the cassette compartment 114 to further ensure that the pistons 133A, 133B align with the fluid pump chambers 138A, 138B of the cassette 112.

The control unit 139 of FIG. 1 is connected to the pressure sensors 151A, 151B, to the stepper motors (e.g., the drivers for the stepper motors) that drive the pistons 133A, 133B, and to the encoders that monitor rotation of the lead screws attached to the stepper motors such that the control unit 139 can receive signals from and transmit signals to those components of the PD system 100. The control unit 139 monitors the components to which it is connected to determine whether any complications exist within the PD system 100, such as the presence of an occlusion or blockage in the patient line 130.

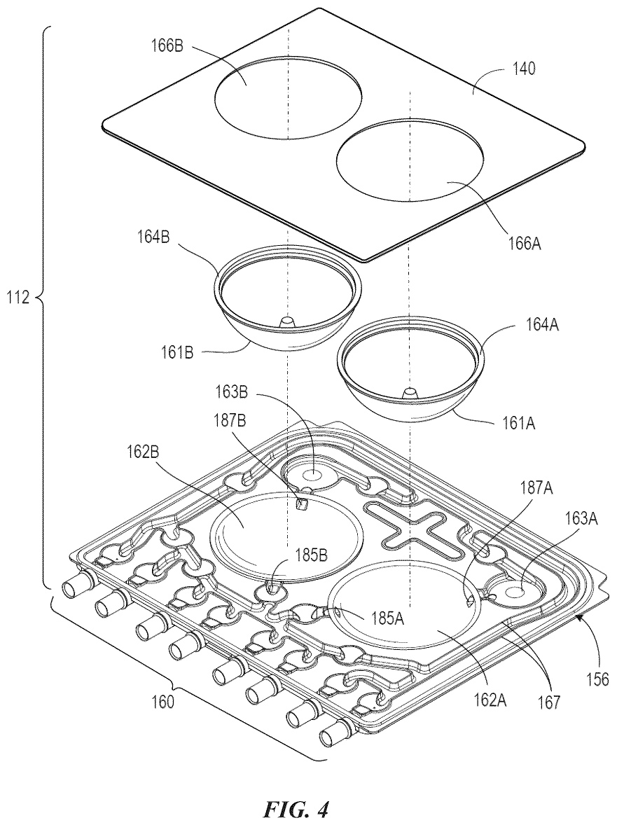

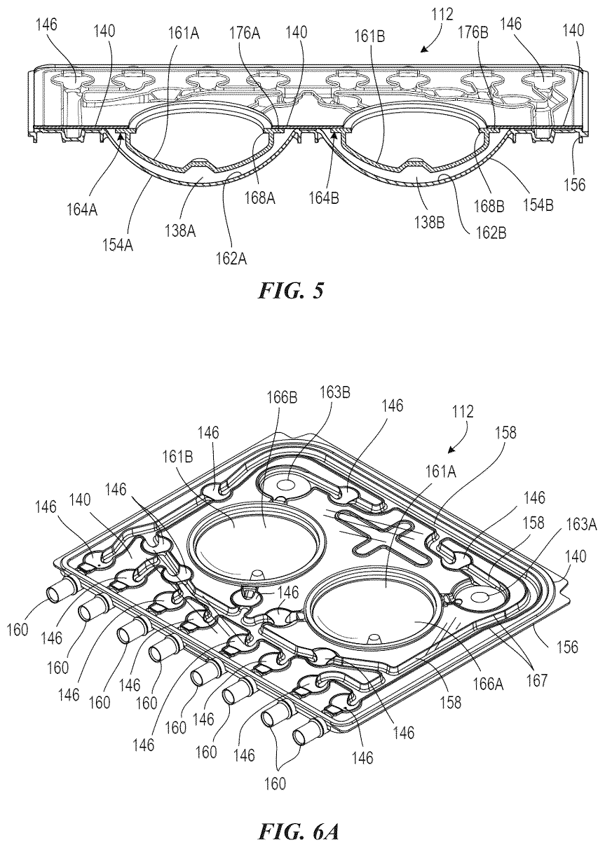

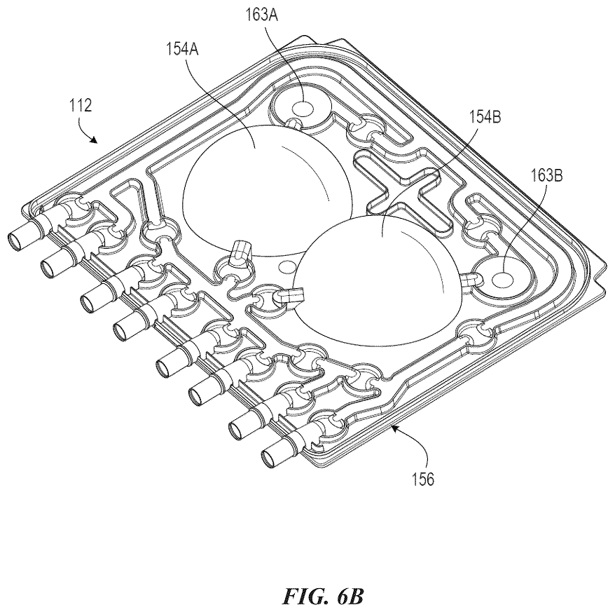

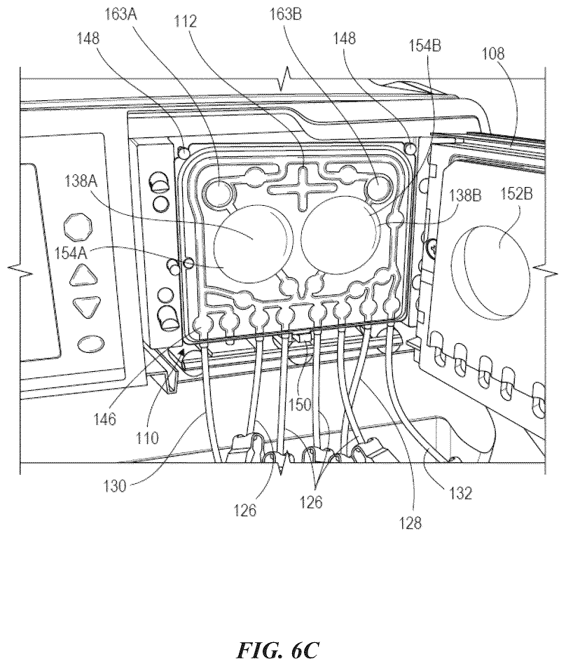

FIG. 4 is an exploded, perspective view of the PD cassette 112 of FIG. 2, in accordance with some embodiments. FIG. 5 is a cross-sectional view of the fully assembled PD cassette 112 of FIG. 2, in accordance with some embodiments. FIGS. 6A and 6B are perspective views of the PD cassette 112 of FIG. 2 from a front side and a back side, respectively, in accordance with some embodiments.

As depicted in FIGS. 4-6B, the PD cassette 112 includes a flexible membrane 140 that is attached to a periphery of a tray-like rigid base 156. Rigid dome-shaped fastening members 161A, 161B are positioned within recessed regions 162A, 162B of the base 156. The dome-shaped fastening members 161A, 161B are sized and shaped to receive the piston heads 134A, 134B of the PD machine 102. In some embodiments, the dome-shaped fastening members 161A, 161B have a diameter, measured from the outer edges of annular flanges 164A, 164B, of about 1.5 inches to about 2.5 inches (e.g., about 2.0 inches) and take up about two-thirds to about three-fourths of the area of the recessed regions 162A, 162B. The annular flanges 164A, 164B of the rigid dome-shaped fastening members 161A, 161B are attached in a liquid-tight manner to portions of the inner surface of the membrane 140 surrounding substantially circular apertures 166A, 166B formed in the membrane 140. The annular flanges 164A, 164B of the rigid dome-shaped fastening members 161A, 161B can, for example, be thermally bonded or adhesively bonded to the membrane 140. The apertures 166A, 166B of the membrane 140 expose the rigid dome-shaped fastening members 161A, 161B such that the piston heads 134A, 134B are able to directly contact and mechanically connect to the dome-shaped fastening members 161A, 161B during use.

The annular flanges 164A, 164B of the dome-shaped fastening members 161A, 161B form annular projections 168A, 168B that extend radially inward and annular projections 176A, 176B that extend radially outward from the side walls of the dome-shaped fastening members 161A, 161B. When the piston heads 134A, 134B are mechanically connected to the dome-shaped fastening members 161A, 161B, the radially inward projections 168A, 168B engage the rear angled surfaces of the sliding latches 145A, 147A of the piston heads 134A, 134B to firmly secure the dome-shaped fastening members 161A, 161B to the piston heads 134A, 1334B. Because the membrane 140 is attached to the dome-shaped fastening members 161A, 161B, movement of the dome-shaped fastening members 161A, 161B into and out of the base 156 (e.g., due to reciprocating motion of the pistons 133A, 133B) causes the flexible membrane 140 to similarly be moved into and out of the recessed regions 162A, 162B of the base 156. This movement allows fluid to be forced out of and drawn into the fluid pump chambers 138A, 138B, which are formed between the recessed regions 162A, 162B of the base 156 and the portions of the dome-shaped fastening members 161A, 161B and membrane 140 that overlie those recessed regions 162A, 162B.

Raised ridges 167 extend from the substantially planar surface of the base 156 towards and into contact with the inner surface of the flexible membrane 140 when the cassette 112 is compressed between the door 108 and the cassette interface 110 of the PD machine 102 to form a series of fluid passageways 158 and to form the multiple, depressible dome regions 146, which are widened portions (e.g., substantially circular widened portions) of the fluid pathways 158. The fluid passageways 158 fluidly connect the fluid line connectors 160 of the cassette 112, which act as inlet/outlet ports of the cassette 112, to the fluid pump chambers 138A, 138B. As noted above, the various inflatable members 142 of the PD machine 102 act on the cassette 112 during use. The dialysate flows to and from the pump chambers 138A, 138B through the fluid pathways 158 and dome regions 146. At each depressible dome region 146, the membrane 140 can be deflected to contact the planar surface of the base 156 from which the raised ridges 167 extend. Such contact can substantially impede (e.g., prevent) the flow of dialysate along the region of the pathway 158 associated with that dome region 146. Thus, the flow of the dialysate through the cassette 112 can be controlled through the selective depression of the depressible dome regions 146 by selectively inflating the inflatable members 142 of the PD machine 102.

The fluid line connectors 160 are positioned along the bottom edge of the cassette 112. As noted above, the fluid pathways 158 in the cassette 112 lead from the pumping chambers 138A, 138B to the various connectors 160. The connectors 160 are positioned asymmetrically along the width of the cassette 112. The asymmetrical positioning of the connectors 160 helps to ensure that the cassette 112 will be properly positioned in the cassette compartment 114 with the membrane 140 of the cassette 112 facing the cassette interface 110. The connectors 160 are configured to receive fittings on the ends of the dialysate bag lines 126, the heater bag line 128, the patient line 130, and the drain line 132. One end of the fitting can be inserted into and bonded to its respective line and the other end can be inserted into and bonded to its associated connector 160. By permitting the dialysate bag lines 126, the heater bag line 128, the patient line 130, and the drain line 132 to be connected to the cassette 112, as depicted in FIGS. 1 & 2, the connectors 160 allow dialysate to flow into and out of the cassette 112 during use. As the pistons 133A, 133B are reciprocated, the inflatable members 142 can be selectively inflated to allow fluid to flow from any of the lines 126, 128, 130, and 132 to any of ports 185A, 185B, 187A, and 187B of the pump chambers 138A, 138B or to allow fluid to flow from any of ports 185A, 185B, 187A, and 187B of the pump chambers 138A, 138B to any of the lines 126, 128, 130, and 132.

The rigidity of the base 156 helps to hold the cassette 112 in place within the cassette compartment 114 of the PD machine 102 and to prevent the base 156 from flexing and deforming in response to forces applied to the projections 154A, 154B by the dome-shaped fastening members 161A, 161B and in response to forces applied to the planar surface of the base 156 by the inflatable members 142. The dome-shaped fastening members 161A, 161B are also sufficiently rigid that they do not deform as a result of usual pressures that occur in the pump chambers 138A, 138B during the fluid pumping process. Thus, the deformation or bulging of the annular portions 149A, 149B of the membrane 140 can be assumed to be the only factor other than the movement of the pistons 133A, 133B that affects the volume of the pump chambers 138A, 138B during the pumping process.

The base 156 and the dome-shaped fastening members 161A, 161B of the cassette 112 can be formed of any of various relatively rigid materials. In some embodiments, these components of the cassette 112 are formed of one or more polymers, such as polypropylene, polyvinyl chloride, polycarbonate, polysulfone, and other medical grade plastic materials. In some embodiments, these components can be formed of one or more metals or alloys, such as stainless steel. These components can alternatively be formed of various different combinations of the above-noted polymers and/or metals/alloys. These components of the cassette 112 can be formed using any of various different techniques, including machining, molding, and casting techniques.

As noted above, the membrane 140 is attached to the periphery of the base 156 and to the annular flanges 164A, 164B of the dome-shaped fastening members 161A, 161B. The portions of the membrane 140 overlying the remaining portions of the base 156 are typically not attached to the base 156. Rather, these portions of the membrane 140 sit loosely atop the raised ridges 165A, 165B, and 167 extending from the planar surface of the base 156. Any of various attachment techniques, such as adhesive bonding and thermal bonding, can be used to attach the membrane 140 to the periphery of the base 156 and to the dome-shaped fastening members 161A, 161B. The thickness and material(s) of the membrane 140 are selected so that the membrane 140 has sufficient flexibility to flex toward the base 156 in response to the force applied to the membrane 140 by the inflatable members 142. In some embodiments, the membrane 140 is about 0.100 micron to about 0.150 micron in thickness. However, various other thicknesses may be sufficient depending on the type of material used to form the membrane 140. Any of various different materials that permit the membrane 140 to deflect in response to movement of the inflatable members 142 without tearing can be used to form the membrane 140. In some embodiments, the membrane 140 includes a three-layer laminate. In some embodiments, inner and outer layers of the laminate are formed of a compound that is made up of 60 percent Septon® 8004 thermoplastic rubber (i.e., hydrogenated styenic block copolymer) and 40 percent ethylene, and a middle layer is formed of a compound that is made up of 25 percent Tuftec® H1062 (SEBS: hydrogenated styrenic thermoplastic elastomer), 40 percent Engage® 8003 polyolefin elastomer (ethylene octane copolymer), and 35 percent Septon® 8004 thermoplastic rubber (i.e., hydrogenated styrenic block copolymer). The membrane 140 can alternatively include more or fewer layers and/or can be formed of different materials.

FIG. 6C illustrates the PD cassette 112 seated against the cassette interface 110, in accordance with some embodiments. As depicted in FIG. 6C, before starting a PD treatment, the door 108 of the PD machine 102 is opened to expose the cassette interface 110, and the cassette 112 is positioned with the dome-shaped fastening members 161A, 161B aligned with the pistons 133A, 133B of the PD machine 102, the pressure sensing chambers 163A, 163B aligned with the pressure sensors 151A, 151B of the PD machine 102, the depressible dome regions 146 aligned with the inflatable members 142 of the PD machine 102, and the membrane 140 adjacent to the cassette interface 110. In order to ensure that the cassette 112 is properly positioned on the cassette interface 110, the cassette 112 is positioned between the locating pins 148 and the spring loaded latch 150 extending from the cassette interface 110. The asymmetrically positioned connectors 160 of the cassette 112 act as a keying feature that reduces the likelihood that the cassette 112 will be installed with the membrane 140 and dome-shaped fastening members 161A, 161B facing in the wrong direction (e.g., facing outward toward the door 108). Additionally or alternatively, the locating pins 148 can be dimensioned to be less than the maximum protrusion of the projections 154A, 154B such that the cassette 112 cannot contact the locating pins 148 if the membrane 140 is facing outward towards the door 108. The pistons 133A, 133B are typically retracted into the piston access ports 136A, 136B during installation of the cassette 112 to avoid interference between pistons 133A, 133B and the dome-shaped fastening members 161A, 161B and, therefore, increase the case with which the cassette 112 can be positioned within the cassette compartment 114.

After positioning the cassette 112 as desired on the cassette interface 110, the door 108 is closed and the inflatable pad within the door 108 is inflated to compress the cassette 112 between the inflatable pad and the cassette interface 110. The compression of the cassette 112 holds the projections 154A, 154B of the cassette 112 in the recesses 152A, 152B of the door 108 and presses the membrane 140 tightly against the raised ridges 167 extending from the planar surface of the rigid base 156 to form the enclosed fluid pathways 158 and dome regions 146. The patient line 130 is then connected to a patient's abdomen via a catheter, and the drain line 132 is connected to a drain or drain receptacle. In addition, the heater bag line 128 is connected to the heater bag 124, and the dialysate bag lines 126 are connected to the dialysate bags 122. At this point, the pistons 133A, 133B can be coupled to the dome-shaped fastening members 161A, 161B of the cassette 112 to permit priming of the cassette 112 and one or more of the lines 126, 128, 130, and 132. Once these components have been primed, the PD treatment can be initiated.

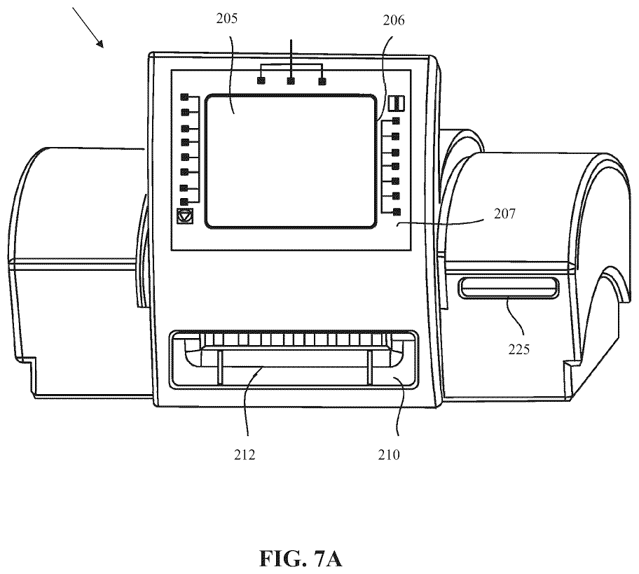

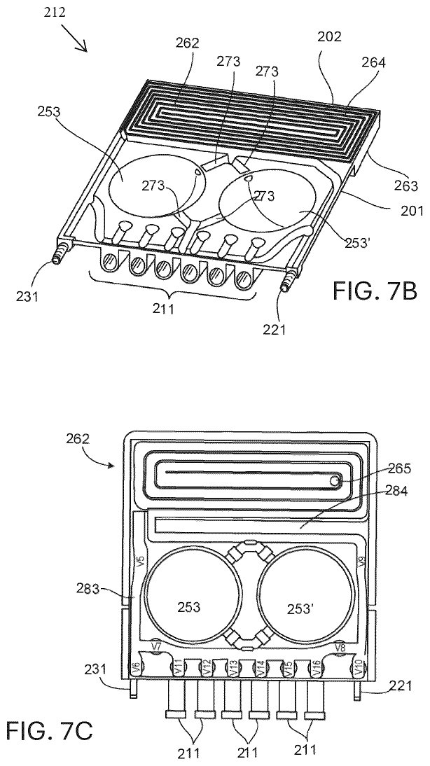

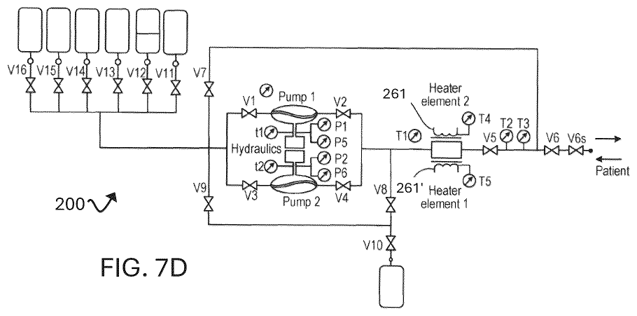

FIG. 7A illustrates a peritoneal dialysis (PD) system including a PD machine 200, and FIGS. 7B and 7C show an embodiment of a cassette 212 operable with the PD machine 200, in accordance with some embodiments. Alternatively to heating of the dialysate via a heating bag as discussed above for PD machine 102, PD machine 200 enables the dialysate to be heated while it is being pumped to the patient. The heating thus works in the form of a continuous-flow water heater which heats the dialysate moved through the fluid system while it is being pumped through the fluid paths. In this concept, a dialysate passage is provided in cassette 212 which is coupled to a heating element of the dialysis machine. While the dialysate flows through the dialysate passage, it takes up heat from the heating element of the dialysis machine while so doing. The PD machine 200 includes a cassette input port 210 into which the cassette 212 may be inserted. A touch screen 205 is provided which allows an interactive menu navigation. Display elements 206 and 207 are also provided which show states and/or other information of the dialysis machine and treatment in compact form. The PD machine 200 may also have a card reader and/or other reader device 225 via which a patient card can be read and/or other data storage device may be inserted. Data on the treatment of the respective patient can be stored on the device 225.

An embodiment of the cassette 212 is shown in FIGS. 7B and 7C. Cassette 212 is similar in many respects to cassette 112. For example, a control unit, similar to control unit 139, provides for control of the valve circuits and other components of PD machine 200. Additionally, cassette 212 has a rigid base 201 of plastic in which the fluid paths and coupling regions are introduced as corresponding cut-outs, chambers and passages. The rigid base can in this respect be produced as an injection molded part or as a deep drawn part. The coupling plane of the rigid base 201 is covered by a flexible film 202 which is welded to the rigid base in a marginal region. During the treatment, the flexible film 202 is pressed with the rigid base by the pressing of the cassette with a coupling surface of the dialysis machine. The fluid paths within the cassette 212 are separated from one another in a fluid tight manner by the pressing of the flexible film with the web regions of the rigid base.

The cassette 212 has connections for the connection of the cassette to the other fluid paths. On the one hand, a connection 221 is provided for the connection to the drain bag 220 as well as a connection 231 for the connection to the connector 230. Corresponding tubing elements (not shown in FIG. 7B) can be provided at these connections. The cassette 212 also has a plurality of connections 211 for the connection of dialysate containers. The connections 211 are designed as connectors to which corresponding connector elements can be connected.

The connections are, in each case, in connection with fluid paths within the cassette. The fluid paths can be opened and closed via valve regions which are numbered consecutively from V1 to V16. In these valve regions, the flexible film 202 can be pressed into the rigid base 201 via valve actuators at the machine side such that the corresponding fluid path is blocked. The cassette 212 in this respect first has a corresponding valve for each connection via which this connection can be opened or closed. A valve V10 is associated with the connection 221 for the drain bag, and a valve V6 is associated with the connection 231 for the patient connector. In some embodiments, a supplemental valve V6s may be further provided to facilitate an operable and safe connection for the patient connector. Valves V11 to V16 are associated with the connections 211 for the dialysate container.

Pump chambers 253 and 253′ are provided in the cassette via which corresponding pump actuators of the dialysis machine can be actuated. The pump chambers 253 and 253′ are concave cut-outs in the rigid base 201 which are covered by the flexible film 202. The film can be pressed into the pump chambers 253 and 253′ or pulled out of these pump chambers again by pump actuators of the dialysis machine. A pump flow through the cassette can hereby be generated in cooperation with the valves V1 to V4 which connect the accesses and outflows of the pump chambers 253 and 253′ and are designated by the reference numeral 273. The pump chambers can in this respect be connected via corresponding valve circuits to all connections of the cassette.

A heating region 262 is also integrated into the cassette 212. In this region, the cassette is brought into contact with or close proximity to heating elements of the dialysis machine which heat the dialysate flowing through this region of the cassette. The heating region 262 in this respect has a passage for the dialysate which extends spirally over the heating region 262. The passage is formed by webs 264 of the rigid base which are covered by the flexible film 202.

The heating region 262 is provided at both sides of the cassette 212. A flexible film is also arranged at the rigid base in the heating region at the lower side 263 of the cassette for this purpose. The flexible film is also welded to the rigid base in a marginal region. A passage is likewise arranged at the lower side and the dialysate flows therethrough. The passages on the lower side and on the upper side are formed by a middle plate of the rigid base which separates the upper side from the lower side and on which webs are downwardly and upwardly provided which form the passage walls. In this respect, the dialysate first flows spirally on the upper side up to the aperture 265 through the middle plate from where the dialysate flows back to the lower side through the corresponding passage. The heating surface which is available for the heating of the fluid can be correspondingly enlarged by the heating region provided at the upper side and at the lower side. Alternatively, the heating region can be arranged on only one side of the cassette.

The cassette 212 also has sensor regions 283 and 284 by which temperature sensors of the dialysis machine can be coupled to the cassette. The temperature sensors in this respect lie on the flexible film 202 and can thus measure the temperature of the liquid flowing through the passage disposed below. Two temperature sensors 284 are arranged at the inlet of the heating region. A temperature sensor 283 via which the temperature of the dialysate pumped to the patient can be measured is provided at the outlet at the patient side.

The dialysate heating in PD system 200 is shown in FIGS. 7B-7D. The heating region 262 is integrated in the cassette in this respect, as was already discussed above. On the coupling of the cassette to the dialysis machine, the heating region 262 of the cassette 212 comes thermally into contact with heating elements of the dialysis machine 200.

The heating elements can in this respect likewise be designed as ceramic heating elements 261, 261′ and can be in contact with heating plates which are coupled to the heating region 262 of the cassette. As shown with respect to the cassette 212, a respective heating plate which heats the dialysate flowing through the heating region is in contact both with the upper side and with the lower side of the heating region.

Sensor regions in the cassette 212 are provided at the inflow and at the outflow of the heating region 262 and come into contact with sensors of the dialysis machine 200 by the coupling of the cassette 212. The temperature of the dialysate flowing into the heating region 262 and the temperature of the dialysate flowing out of the heating region 262 can thus be determined by the temperature sensors T1 to T3 (FIG. 7D). Temperature sensors T4 and T5 may also be provided to determine the temperature of the heating elements and/or of the heating plates. Pressure sensors (P1, P2, P5, P6) and temperature sensors (t1, t2) may be provided to provide information concerning the hydraulic operation of the cassette and pumps (Pump 1, Pump 2).

To enable a coupling of the actuators and/or sensors of the dialysis machine 200 to the corresponding regions of the cassette 212, the dialysis machine 200 has a cassette receiver with a coupling surface to which the cassette 212 can be coupled. The corresponding actuators, sensors and/or heating elements of the dialysis machine 200 are arranged at the coupling surface. The cassette 212 is pressed with this coupling surface such that the corresponding actuators, sensors and/or heating elements come into contact with the corresponding regions in the cassette 212.

In this respect, a mat of a flexible material, such as a silicone mat, is advantageously provided at the coupling surface of the dialysis machine 200. It ensures that the flexible film of the cassette 212 is pressed with the web regions of the cassette 212 and thus separates the fluid paths within the cassette 212.

A peripheral margin of the coupling surface is advantageously provided which is pressed with the marginal region of the cassette 212. The pressing in this respect advantageously takes place in an airtight manner so that an underpressure or vacuum can be built up between the coupling surface and the cassette.

A vacuum system can optionally be provided to pump air out of the space between the coupling surface and the cassette 212. A particularly good coupling of the actuators, sensors and/or heating elements of the peritoneal dialysis device 200 with the corresponding regions of the cassette 212 is hereby made possible. In addition, the vacuum system allows a leak tightness check of the cassette 212. A corresponding vacuum is applied after the coupling for this purpose and a check is made whether this vacuum is maintained.

The compression of the cassette 212 against the coupling surface of the dialysis machine 200 can take place pneumatically, for example. For this purpose, usually an air cushion is provided which is filled with compressed air and thus presses the cassette 212 onto the coupling surface.

The cassette receiver usually has a receiver surface which is disposed opposite the coupling surface and into which the rigid base of the cassette 212 is inserted. The receiver surface advantageously has corresponding recesses for this purpose. The receiver surface with the inserted cassette can then be pressed onto the coupling surface via a pneumatic pressing apparatus.

The insertion of the cassette 212 can take place in different ways. In the dialysis machine 200, a drawer 210 can be moved out of the dialysis machine 200 to receive the cassette 212. The cassette 212 is inserted into this drawer 210. The cassette 212 is then pushed into the dialysis machine 200 together with the drawer 210. The pressing of the cassette 212 with the coupling surface which is arranged in the interior of the dialysis machine 200 is carried out by moving the cassette 212 and the coupling surface mechanically toward one another and then pressing them together pneumatically.

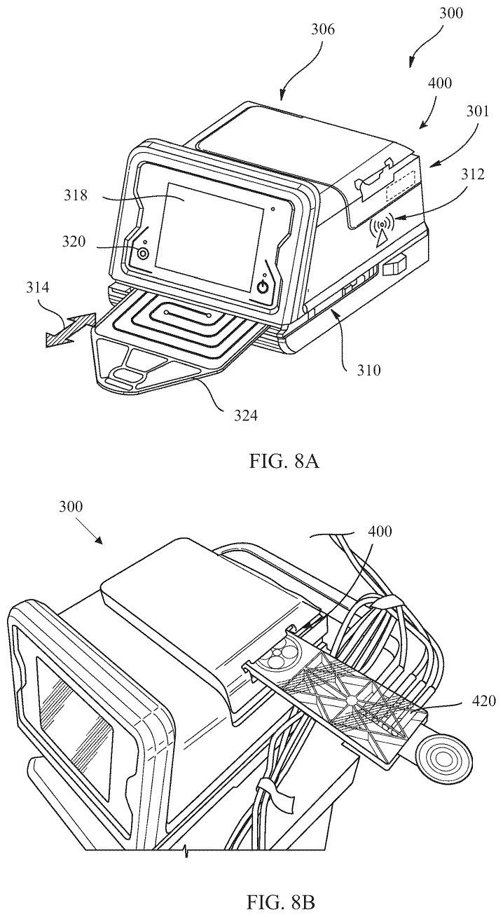

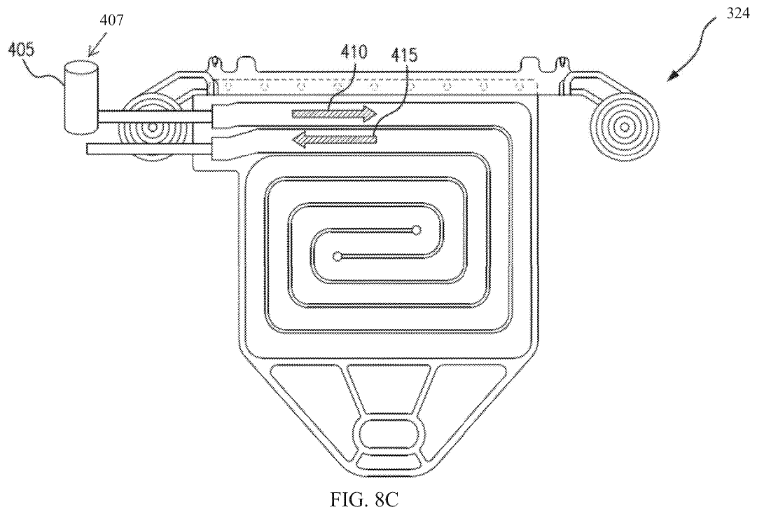

FIG. 8A illustrates a peritoneal dialysis (PD) system including a PD machine 300, and FIGS. 8B and 8C show an embodiment of a dialysis cassette 420 and a heating cassette 324 operable with PD machine 300, in accordance with some embodiments. Alternatively to heating of the dialysate via a heating bag as discussed above for PD machine 102, PD machine 300 enables the dialysate to be heated while it is being pumped to the patient, e.g., for example using a peristaltic pump internal to PD machine 300. The heating thus works in the form of a continuous-flow water heater which heats the dialysate moved through the fluid system while it is being pumped through the fluid paths.

As shown in FIG. 8A, the dialysis machine 300 may include, for example, a housing 306, a processing module 301, a data connection component 312, a touch screen 318, and a control panel 320 operable by a user (e.g., a caregiver or a patient) to allow, for example, set up, initiation, and/or termination of a dialysis treatment. The touch screen 318 and the control panel 320 may allow a user to input various treatment parameters to the dialysis machine 300 and to otherwise control the dialysis machine 300. In addition, the touch screen 318 may serve as a display. The touch screen 318 may function to provide information to the patient and the operator of the dialysis machine 300. For example, the touch screen 318 may display information related to a dialysis treatment to be applied to the patient, including information related to a prescription.

The dialysis machine 300 may include a processing module 301 that resides inside the dialysis machine 300, the processing module 301 being configured to communicate with the touch screen 318 and the control panel 320. The processing module 301 may be configured to receive data from the touch screen 318, the control panel 320, and sensors, e.g., air, temperature and pressure sensors, and control the dialysis machine 300 based on the received data. For example, the processing module 301 may adjust the operating parameters of the dialysis machine 300, including control of valve and pump operations like that discussed elsewhere herein.

The dialysis machine 300 may be configured to connect to a network. The connection to network may be via a wired and/or wireless connection. The dialysis machine 300 may include a connection component 312 configured to facilitate the connection to the network. The connection component 312 may be a transceiver for wireless connections and/or other signal processor for processing signals transmitted and received over a wired connection. Other medical devices (e.g., other dialysis machines) or components may be configured to connect to the network and communicate with the dialysis machine 300.

The PD machine 300 includes a cassette port 400 that is arranged and configured to receive the cassette 420. The cassette 420 may be insertable into the cassette port 400 formed in the PD machine 300. In an embodiment, a control unit, similar to control unit 139, provides for control of the valve circuits, pump(s) and other components of PD machine 300.

As illustrated in FIG. 8B, in one embodiment, the cassette port 400 is arranged horizontally in the PD machine 300 (e.g., extending across the PD machine 300 between side surfaces). In one embodiment, the cassette port 400 may extend from a side surface of the PD machine 300. In use, the cassette 420 may be connected to dialysate bag lines, which may be used to pass dialysate from dialysate bags to the cassette 420. In use, the cassette 420 may be disposable. Alternatively, the cassette 420 may be reusable. Thus arranged, with the cassette 420 positioned in the cassette port 400, the at least one pump (not shown) positioned within the PD machine 300 may be operated to pump fluid, e.g., fresh and spent dialysate, to and from the patient. For example, the pump may transfer dialysate from the dialysate bag(s) through, for example, the cassette 420 inserted into cassette port 400 in the dialysis machine, to a heating chamber (not shown) prior to transferring the dialysis to the patient. In an embodiment, the pump may be a peristaltic pump.

The dialysate may need to be heated to body temperature prior to being inserted into the patient (e.g., it is preferred that dialysate should be delivered to patients at specific temperatures, for example, at 37° Celsius (e.g., body temperature)). The PD machine 300 may also include one or more heating elements disposed internal to the machine 300 and an opening or cavity 310 (used interchangeably herein without the intent to limit) arranged and configured to receive the heating cassette 324 in a direction indicated at arrow 314. In use, the heating cassette 324 may be inserted into the opening 310 formed in the PD machine 300 and into the heating chamber positioned within the dialysis machine 300. In some embodiments, the heating cassette 324 may be configured so dialysate may continually flow through the heating cassette 324 to achieve a predetermined temperature before flowing into the patient. For example, in some embodiments the dialysate may continually flow through the heating cassette 324 at a rate of approximately 300 ml/min. Thus arranged, the pump may pump dialysate from the dialysate bag(s) through, for example, the cassette 420 positioned in the cassette port 400, through the heating cassette 324 positioned in the heating chamber, and eventually to the patient.

In use, with the heating cassette 324 inserted into the cavity 310, the one or more heating elements may affect the temperature of dialysate flowing through the heating cassette 324. In some embodiments, the heating chamber may be arranged and configured so that a portion of tubing in the system is passed by, around, or otherwise configured with respect to, one or more heating elements. In some embodiments, a dialysis machine 300 may provide an active measurement of the dialysate temperature in dialysate bags and/or a heating chamber, e.g., in the dialysate bags, and the heating chamber. It is understood that FIGS. 8A and 8B illustrate dialysate continuously flowing through the heating cassette 324 “in-line” with the dialysis machine 300, reaching an acceptable temperature by the application of internal heating elements.

As shown in FIG. 8C, the heating cassette 324 may include a filter 405 in-line with heater cassette 324, e.g., coupled to an inlet of the warmer cassette 324. For example, dialysate flowing to the patient through the heater cassette 324 from dialysate bags may flow through the filter 405, e.g., before entering the heater cassette 324. In embodiments, the filter 405 may be coupled to the heater cassette 324 directly or indirectly by tubing. The filter 405 may filter out air content in the dialysate flow. In some embodiments, the filter 405 may be a container 407, e.g., a cylindrical container, having an inlet for receiving dialysate and air, and an outlet for flowing dialysate with air content filtered out of the dialysate. It is understood that the container 407 may be any configuration, e.g., size and/or shape, to filter air content from dialysate. In some embodiments, the filter 405 may be arranged as part of a patient connector (for example, as part of a cap of the patient connector). In various embodiments, a filter in addition to filter 405 may be arranged as part of a patient connector. For example, warmer cassette 324 may include a hydrophobic filter (not shown) at the end of the patient line through which air may be expelled during priming.

Dialysate may flow through the filter 405 at the inlet of the heater cassette 224 and may flow through an extended flow path in the heater cassette 324. For example, a flow path may be a tortuous, or circuitous, pathway, so that the dialysate may flow at a constant rate into the patient and may heat to the desired predetermined temperature while flowing through the tortuous flow path of the heater cassette 324. The dialysate may flow from the warmer cassette into the patient at an outlet of the heater cassette 324, indicated at arrow 415. Although the flow path shown in FIG. 8C is somewhat circular, any labyrinth of circuitous flow path may be incorporated in the heater cassette 324 to ensure a constant flow of the dialysate so that the dialysate temperature is heated to the predetermined temperature before flowing into the patient. As further discussed in detail elsewhere herein, in some embodiments, the heater cassette 324 may further be used, at least in part, in connection with mixing operations for on-site mixing of dialysate solutions.

U.S. Pat. Nos. 9,867,921 B2, 11,426,502 B2 and 11,413,386 B2, which are incorporated by reference herein, disclose additional features of PD machines and components useful herein.

### On-Site Dialysate Generation

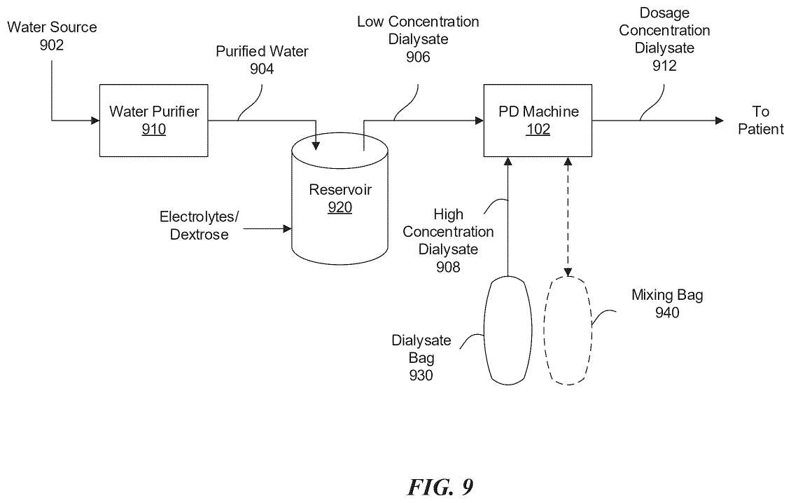

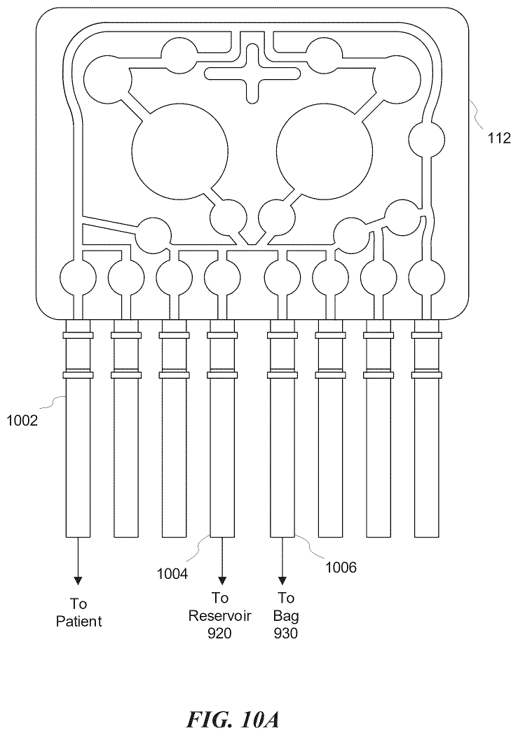

FIG. 9 illustrates a system 900 for producing dialysate solution at the point of care, in accordance with some embodiments. Production of dialysate solution is typically performed by a manufacturer and stored in sterile bags that are then shipped to the consumer (e.g., healthcare provider, patient, etc.) to be used for one or more PD treatments. The dialysate solution is typically mixed at one or more pre-set concentrations, by weight, of dextrose (or glucose) to water. In addition, the dialysate solution may contain common electrolytes such as sodium, potassium, calcium, magnesium, chloride, and/or bicarbonate. However, the solutions are typically only made available at a fixed set of concentrations (e.g., 1.5%, 3%, and 4.5% dextrose). Thus, healthcare professionals may prescribe a PD treatment for a patient using dialysate solution having one of the available concentrations of dextrose that are provided by the manufacturer. The dialysate solution is then shipped to the point of care for treatment.

This has a number of drawbacks. First, the vast majority of weight and volume being shipped consists of purified water, which is heavy. Shipping costs are therefore high to ship the dialysate solution to the point of care. In addition, a single patient may go through tens or hundreds of liters of dialysate solution in a week, requiring recurring shipments of tens or hundreds of pounds of dialysate every two weeks. Since most point of care locations have access to a water source, the amount of dialysate shipped to the patient can be reduced if high concentration dialysate can be diluted with purified water on-site at the point of care.

In accordance with some embodiments, a system 900 is provided that makes use of a modified PD machine 102 or 200 or 300 to mix a high-concentration dialysate solution, which may be referred to herein as concentrated dialysate solution, with a low-concentration dialysate solution, which may be referred to herein as a base dialysate solution. In an embodiment, the system 900 includes a water purifier 910, a reservoir 920, and the PD machine 102 or 200 or 300.

A water source 902, such as tap water, pre-filtered water, distilled water, or the like is commonly available at the point of care. However, the water source 902 cannot be used directly to dilute the concentrated dialysate solution due to concerns about patient safety due to contaminants (e.g., chemical, biological, etc.) that may be present in the water source 902. The water purifier 910 is used to treat the water source 902 to generate purified water 904.

In an embodiment, the water purifier 910 includes one or more of a mechanical filter, an activated carbon filter, a reverse osmosis membrane, or a deionization filter. The mechanical filter may include a sand filter, cartridge filter, or the like. In some embodiments, two or more mechanical filters may be used in series, with each filter being rated to filter out particles of different sizes. In some embodiments, the water purifier 910 may also include chemical filters such as an activated carbon filter, which are suited for removing some organic chemicals from the mechanically filtered water. Other types of substrate may also be used in lieu of activated carbon.

In some embodiments, the water purifier 910 may include a reverse osmosis (RO) membrane. The RO membrane is particularly suited to remove salts and other pollutants from the filtered water that may not have been filtered out via the mechanical filters and/or chemical filters. In some embodiments, a deionization filter may be included in the water purifier 910 after the RO membrane. The deionization (DI) filter may attract and remove some minerals or other dissolved solids in the water that are attracted to the resin beads in the DI filter.

Although the filtered water that has passed through the RO membrane and DI filter may have very low or undetectable levels of total dissolved solids (TDS), this water may still have some additional biological contaminants. Bacteria in the water may release endotoxins (lipopolysaccharides), Ribonuclease (RNase), and/or Deoxyribonuclease (DNase) that could be harmful to the patient if introduced to the peritoneal cavity. Therefore, in some embodiments, the water purifier 910 may also include a sterilization component such as a UV light source or endotoxin (Ultra) filter capable of oxidizing or removing these harmful contaminants.

The water filter 910 may include any system well known in the art for filtering, purifying, and/or sterilizing water. The water filter 910 thereby produces purified water 904 that can be collected in a reservoir 920. In an embodiment, the reservoir 920 is larger than most dialysate bags available on the market and, therefore, the total volume of base dialysate solution 906 that can be produced and stored in the reservoir 920 is greater than that of a conventional dialysate bag. For example, in an embodiment, the reservoir 920 may have a volume of 60 liters, which is enough base dialysate solution for 5 days or more of PD treatments.

In an embodiment, the reservoir 920 may be emptied and a fixed amount of electrolytes and/or dextrose may be added to the reservoir 920. The fixed amount may be measured, by weight, to provide a target concentration of dextrose when a given volume of purified water 904 is added thereto. In one embodiment, the amount of dextrose added to the reservoir corresponds to the desired concentration of the base dialysate solution 906. For example, to get a 1.5% concentration, by weight, solution of dextrose, 15 grams of dextrose are added to the reservoir 920 for each liter of purified water 904 (˜1 kg). Thus, for a 60 liter volume reservoir 920, 900 grams of dextrose may be added to the reservoir 920 and mixed with 60 liters of purified water 904.

In some embodiments, the base dialysate solution 906 contains zero dextrose. In such cases, the base dialysate solution may contain purified water 904 and some concentration of electrolytes. In other words, the base dialysate solution 906 has a dextrose concentration of 0.0%, whereas the electrolyte concentration may be greater than 0%.

Once a volume of base dialysate solution is mixed in the reservoir 920, a PD treatment can be performed using the PD machine 102 or 200 or 300. The PD machine 102 or 200 or 300 may have components similar to prior art PD machines. However, the PD machine 102 or 200 or 300 may be modified, e.g., via a software update and/or by physical components and connections according to that described herein, to operate in a manner that mixes the base dialysate solution 906 with the concentrated dialysate solution 908. In an embodiment, a disposable cassette 112 or 212, or in some embodiments a cassette 324 or 420, is connected, via a first fluid line connected to a first port of the disposable cassette 112 or 212, 324, 420, to the reservoir 920, allowing the PD machine 102 or 200 or 300 to draw the base dialysate solution 906 from the reservoir 920 using the one or more pumps discussed herein. The disposable cassette 112 or 212 or or 324 or 420 is also connected, via a second fluid line connected to a second port of the disposable cassette 112 or 212 or 324 or 420, to a dialysate bag 930 that contains a high-concentration dextrose solution, referred to herein as the concentrated dialysate solution 908. The concentrated dialysate solution 908 may be, e.g., 50% dextrose by weight. It will be appreciated that any concentration of dextrose that is greater than the intended dosing concentration may be used as the concentrated dialysate solution. Lower concentrations (<50%) will use a lower ratio of base dialysate solution to concentrated dialysate solution to generate the desired target dosage dialysate solution 912. Higher concentrations (>50%) may be oversaturated because the solubility of dextrose in water is approximately 450 g/L at room temperature. In some cases, the dialysate bag 930 may be heated (e.g., using a heater element of the PD machine 102 or dialysate may be heated inline in PD machine 200, 300), thereby raising the solubility of dextrose in the bag 930 during treatment and allowing for a slightly higher concentration (e.g., ˜>50% by weight) of dextrose to be used while ensuring the dextrose is fully dissolved in the solution.

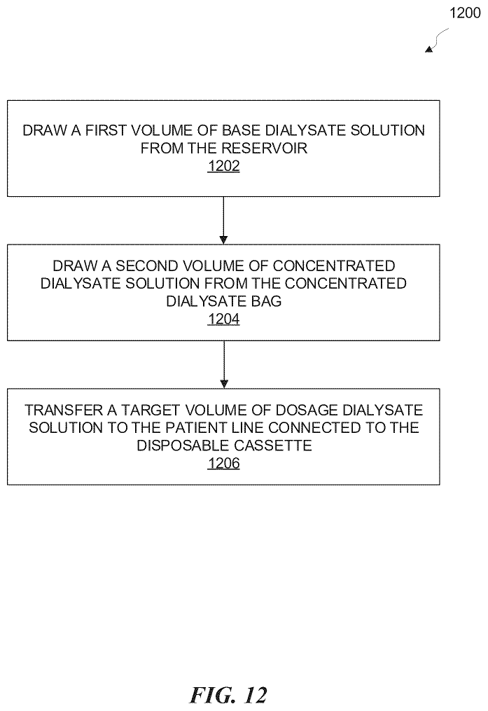

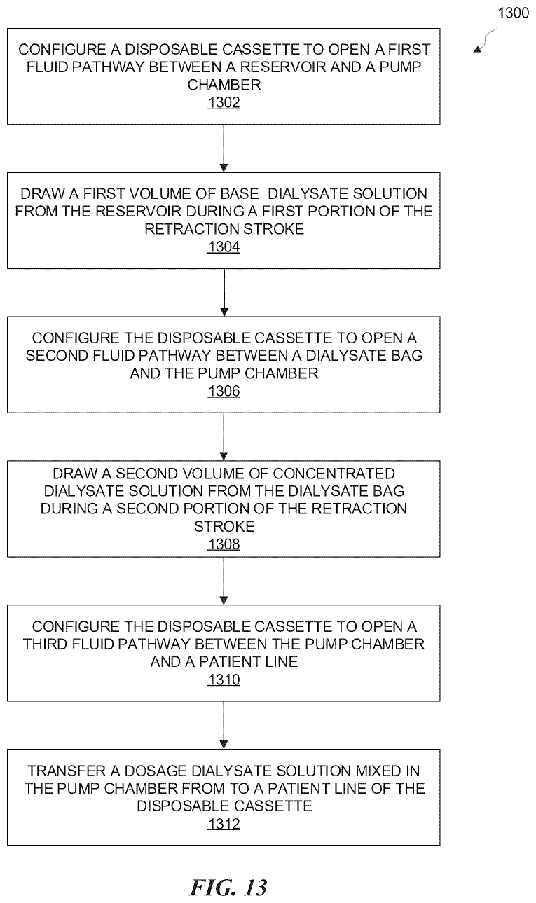

In some embodiments, the mixing is accomplished dynamically in smaller increments of the total fill volume by mixing the two solution components in at least one of one or more pump chambers of the disposable cassette 112 or 212 and/or a fluid line (e.g., patient line) connected to the disposable cassette 112 or 212 or 324 or 420. More details about the different mixing modes will be set forth below with reference to FIGS. 12-13. In other embodiments, a mixing bag 940 may be attached to a third fluid line connected to a third port of the disposable cassette 112 or 212 or 324 or 420. The mixing bag 940 may be used as a secondary reservoir to combine the base dialysate solution 906 with the concentrated dialysate solution 908 prior to transferring the target dialysate solution 912 to the patient. In such embodiments, a first volume (e.g., 1.9 L @ 0% dextrose concentration by weight) of base dialysate solution 906 is drawn from the reservoir 920 and transferred to the mixing bag 940. Then, a second volume (e.g., 100 mL @ 50% dextrose concentration by weight) of concentrated dialysate solution 908 is drawn from the dialysate bag 930 and transferred to the mixing bag 940. The resulting target dialysate solution 912 in the mixing bag 940 has a total volume of 2 L and a concentration of 2.5% dextrose by weight.

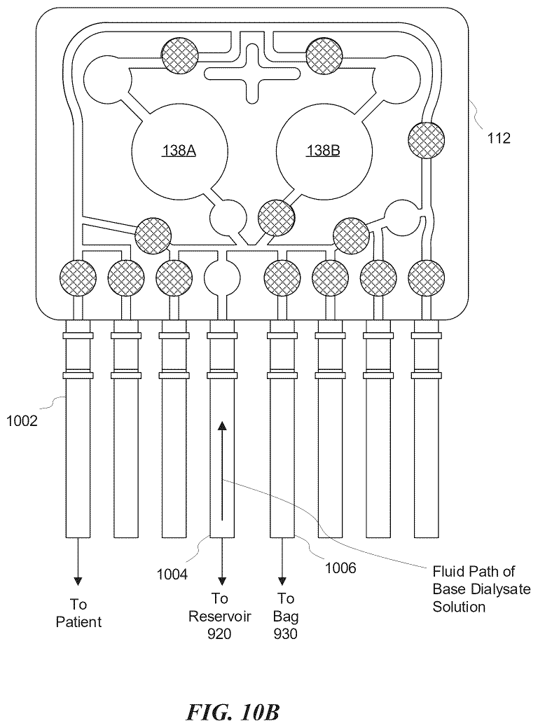

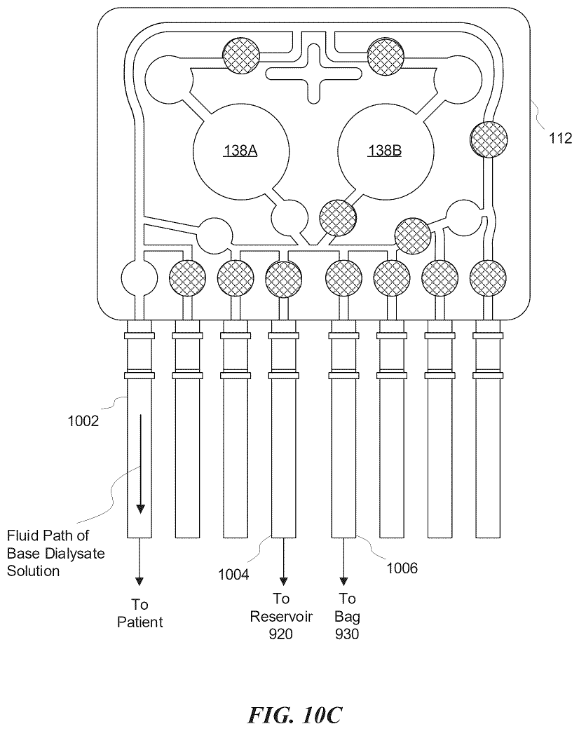

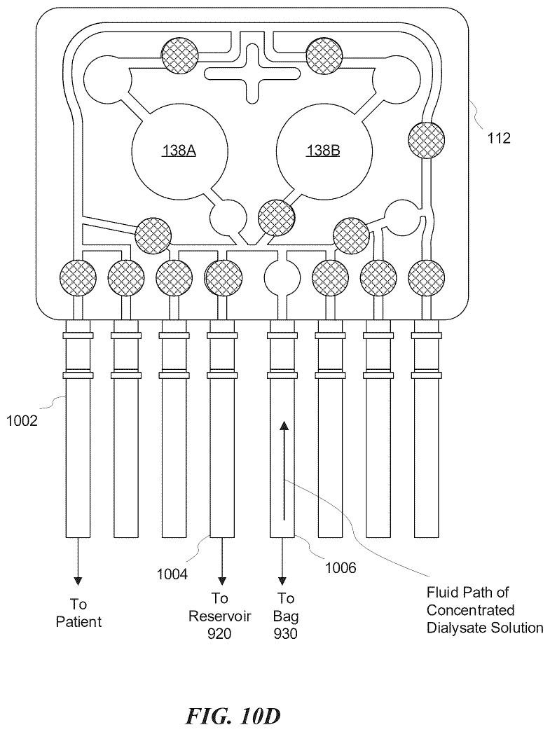

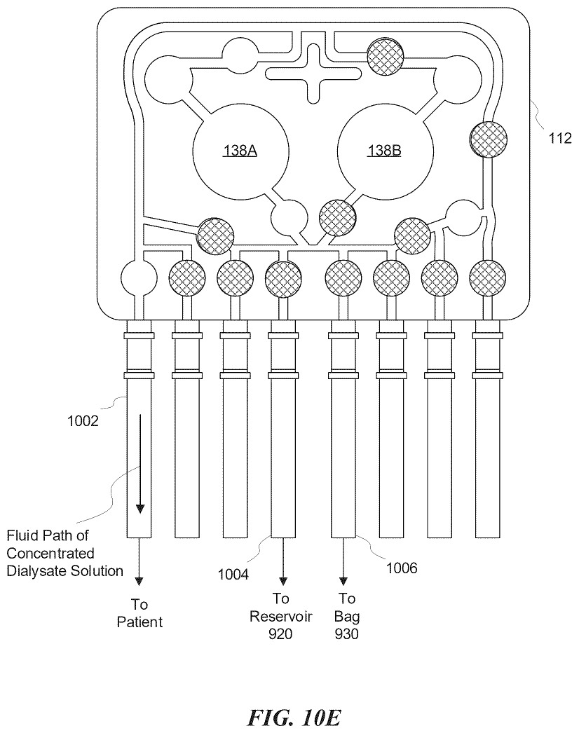

FIGS. 10A-10F are conceptual illustrations of the fluid pathways for accomplishing mixing of dialysate solution to a final target dosage concentration in the disposable cassette 112, in accordance with some embodiments (mixing in disposable cassette 212 may be performed in a similar fashion). A top view of the disposable cassette 112 is shown having a number of dialysate fluid lines 126 connected thereto via a number of ports of the disposable cassette 112. The fluid lines 126 include a patient line 1002 connecting the fluid pathways of the disposable cassette 112 to a catheter disposed in a peritoneal cavity of the patient. The fluid lines 126 also include a first fluid line connected to the reservoir 920 and a second fluid line 1006 connected to the dialysate bag 930 containing the concentrated dialysate solution 908.

It will be appreciated that the two large circles represent the pump chambers 138, the two intermediate circles represent the pressure sensing chambers 163, and the 16 smaller circles represent the depressible dome regions that interact with the inflatable members 142 to open and close fluid pathways in the disposable cassette 112. The pumps (or pistons 133/piston heads 134) interact with the pump chambers 138 to increase or decrease pressure in the pump chambers 138 to force fluid out of the pump chambers 138 or draw fluid into the pump chambers 138, in accordance with one or more open fluid pathways in the disposable cassette 112.

FIG. 10B shows a first configuration of the disposable cassette 112 for drawing base dialysate solution 906 from the reservoir 920 into a pump chamber 138A of the disposable cassette 112. The cross-hatched depressible dome members are closed by inflating the corresponding inflatable members 142 located in the cassette interface 110. This creates a fluid pathway from the first fluid line 1004 to the first pump chamber 138A. By moving the piston 133A in a retraction stoke, the volume of the pump chamber 138A expands, thereby lowering the pressure within the pump chamber 138A, which draws fluid from the reservoir 920 into the pump chamber 138A.

FIG. 10C shows a second configuration of the disposable cassette 112 for forcing dialysate solution from the first pump chamber 138A to the patient line 1002. Once a first volume of fluid has been transferred from the reservoir 920 to the pump chamber 138A, the configuration of the disposable cassette 112 is changed to close the fluid pathway between the reservoir 920 and the pump chamber 138A, and open a fluid pathway between the pump chamber 138A and the patient line 1002. The piston 133A reverses direction in a compression stroke and the volume of the pump chamber 138A is reduced, thereby increasing the pressure within the pump chamber 138A, which forces the fluid from the pump chamber 138A into the patient line 1002.

FIG. 10D shows a third configuration of the disposable cassette 112 for drawing concentrated dialysate solution 908 from the bag 930 into the pump chamber 138A of the disposable cassette 112. The cross-hatched depressible dome members are closed by inflating the corresponding inflatable members 142 located in the cassette interface 110. This creates a fluid pathway from the second fluid line 1006 to the first pump chamber 138A. By moving the piston 133A in a retraction stoke, the volume of the pump chamber 138A expands, thereby lowering the pressure within the pump chamber 138A, which draws fluid from the bag 930 into the pump chamber 138A.

FIG. 10E shows a fourth configuration of the disposable cassette 112 for forcing concentrated dialysate solution from the first pump chamber 138A to the patient line 1002. Once a second volume of fluid has been transferred from the bag 930 to the pump chamber 138A, the configuration of the disposable cassette 112 is changed to close the fluid pathway between the bag 930 and the pump chamber 138A, and open a fluid pathway between the pump chamber 138A and the patient line 1002. The piston 133A reverses direction in a compression stroke and the volume of the pump chamber 138A is reduced, thereby increasing the pressure within the pump chamber 138A, which forces the fluid from the pump chamber 138A into the patient line 1002.

It will be appreciated that the fluid pathways of FIGS. 10C and 10E may be used interchangeably to transfer both the base dialysate solution 906 and the concentrated dialysate solution 908 to the patient line 1002. Furthermore, it will be readily apparent that the second pump chamber 138B can be used similarly to how the first pump chamber 138A is used by re-configuring the inflatable members to close off and/or open various depressible dome regions of the disposable cassette 112.

Furthermore, it will be appreciated that the configurations of the disposable cassette 112 or 212 or 324 or 420 illustrate configurations that may be used with multiple types of filling modalities, which will be described in more detail below. Finally, the configuration of fluid pathways in the disposable cassette 112 or 212 or 324 or 420 is only one possible implementation of the disposable cassette 112 or 212 or 324 or 420 and other embodiments of the disposable cassette 112 or 212 or 324 or 420 with more or fewer ports, depressible dome members, pump chambers, or the like are contemplated as being within the scope of the present disclosure.

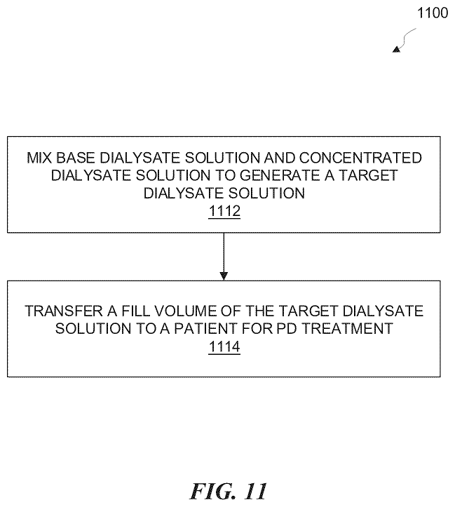

FIG. 11 is a flow diagram of a method 1100 for operating a peritoneal dialysis machine, in accordance with some embodiments. It will be appreciated that the method 1100 is described as being performed by the PD machine 102 or 200 or 300. More specifically, the various steps described below can be implemented by a processor, such as implemented within the control unit 139 of the PD machine 102 or 200 or 300, configured to execute a number of instructions. In various embodiments, the method 1100 can be implemented using hardware, software executed by at least one general purpose processor configured to control a specialized apparatus such as a PD machine, or a combination of hardware and software. The following will describe the method 1100 with specific reference to PD machine 102, although the method when using PD machine 200 with cassette 212 will operate in a similar fashion. Further, aspects of the method 1100 may be appropriately incorporated in connection with pumping operations using a peristaltic pump and mixing of concentrates using a mixing bag, for example, like that described in connection with the features and components of the PD machine 300.

At step 1112, the PD machine 102 mixes a first volume of the base dialysate solution at a first concentration from the reservoir with a second volume of concentrated dialysate solution at a second concentration to create a target dosage dialysate solution at a third concentration between the first concentration and the second concentration. In an embodiment, the mixing is performed in a number of smaller amounts compared to a total fill volume of a PD treatment cycle. For example, if a total of 1 L of dialysate is to be transferred to the patient, 5-20 mL of target dosage dialysate solution may be mixed at a time in at least one of a pump chamber of the disposable cassette 112 and/or a fluid line connected to the disposable cassette 112. The mixing process is repeated N times until a total volume of target dosage dialysate solution has been mixed and transferred to the patient. Various modalities for the mixing step 1112 can be implemented by the PD machine 102.

At 1114, the PD machine 102 transfers the target dosage dialysate solution 912 to the patient in a fill phase of a PD treatment cycle. The PD machine 102 will then wait for a dwell time period before draining the effluent from the patient.

FIG. 12 is a flow diagram of a method 1200 for mixing two dialysate solutions using a first mixing modality, in accordance with some embodiments. The following will describe the method 1200 with specific reference to PD machine 102, although the method when using PD machine 200 with cassette 212 will operate in a similar fashion. The method 1200 may be performed utilizing full strokes of the piston 133 of the pump mechanism.

At 1202, the PD machine 102 draws a first volume of base dialysate solution 906 from the reservoir 920 using the disposable cassette 112. In an embodiment, each full retraction stroke of the piston 133 will result in a fixed volume of fluid being drawn into the pump chamber 138, wherein the fixed volume is less than the first volume. Thus, in order to draw the first volume of base dialysate solution 906 from the reservoir 920, a series of N full strokes of the piston 133 are performed, alternating the fluid pathway of the disposable cassette 112 between each retraction or compression stroke to alternately draw fluid from the reservoir 920 and transfer fluid to at least one of the patient line 1002 and/or the mixing bag 940.

At 1204, the PD machine 102 draws a second volume of concentrated dialysate solution 906 from the dialysate bag 930 using the disposable cassette 112. In an embodiment, each full retraction stroke of the piston 133 will result in a fixed volume of fluid being drawn into the pump chamber 138, wherein the fixed volume is less than or equal to the second volume. Thus, in order to draw the second volume of concentrated dialysate solution 908 from the bag 930, a series of M full strokes of the piston 133 are performed, alternating the fluid pathway of the disposable cassette 112 between each retraction or compression stroke to alternately draw fluid from the dialysate bag 930 and transfer fluid to at least one of the patient line 1002 and/or mixing bag 940.

It will be appreciated that the ratio of N/M is selected to get a target concentration of the target dosage dialysate solution to be transferred to the patient. Since the fixed volume associated with a full stroke of the piston 133 is equal for both the base dialysate solution and the concentrated dialysate solution, the target concentration (cₜ) is given by:

$${c}_{t} = \frac{{c}_{1} {V}_{1} + {c}_{2} {V}_{2}}{{V}_{1} + {V}_{2}} , \qquad ( Eq . 1 )$$

where c₁ is the concentration of dextrose in the base dialysate solution, c₂ is the concentration of dextrose in the concentrated dialysate solution, V₁ is the first volume of the base dialysate solution, V₂ is the second volume of the concentrated dialysate solution, and the ratio of

$$\frac{{V}_{1}}{{V}_{2}} = \frac{N}{M} .$$

At 1206, the PD machine 102 transfers (e.g., fills) a fill volume of the target dosage dialysate solution to a patient line connected to the disposable cassette 112.

It will be appreciated the steps 1202, 1204, and 1206 can be divided into smaller subsets of the total volumes and interspersed as necessary. For example, the PD machine 102 can draw N fixed volumes of base dialysate solution from the reservoir 920 and transfer, after each retraction stroke of the piston 133, each of the N fixed volumes of base dialysate solution to the patient line during the corresponding compression stroke of the piston 133. Then, the PD machine 102 can draw M fixed volumes of concentrated dialysate solution from the dialysate bag 930 and transfer, after each retraction stroke of the piston 133, each of the M fixed volumes of concentrated dialysate solution to the patient line during the corresponding compression stroke of the piston 133. Thus, the steps 1202, 1204, and 1206 may be partially performed and repeated a number of times, intermittently.

In accordance with another modality, the method 1200 can be performed using partial strokes of the piston 133. For example, when the piston 133 is actuated by, e.g., a stepper motor, it is relatively simple to adjust the total length of the stroke, thus changing the fixed volume of fluid drawn into or forced out of the pump chamber 138 during each partial stroke. Consequently, method 1200 can be performed using partial strokes (e.g., ½, ¼, 30%, etc.) of the piston 133 to adjust the fixed volume associated with each stroke. This can allow for more fine adjustment of the target concentration of the target dosage dialysate solution. By utilizing more strokes at smaller fixed volumes, it is easier to get closer to any desired target concentration. It will be appreciated that in the method 1200, either using full strokes (e.g., where the fixed volume is the maximum volume that can be drawn into the pump chamber 138) or partial strokes (e.g., where the fixed volume is less than the maximum volume that can be drawn into the pump chamber 138), the base dialysate solution and the concentrated dialysate solution are not mixed within the pump chamber 138, but instead are at least partially mixed in the patient line (or alternately the mixing bag 940) and finally mixed within the peritoneal cavity of the patient.

FIG. 13 is a flow diagram of a method 1300 for mixing two dialysate solutions in accordance with another mixing modality, in accordance with some embodiments. The following will describe the method 1300 with specific reference to PD machine 102, although the method when using PD machine 200 with cassette 212 will operate in a similar fashion. The method 1300 may be performed by dividing a full stroke of the piston 133 of the pump mechanism into two distinct stages, drawing base dialysate solution into the pump chamber during the first stage of the retraction stroke and then drawing concentrated dialysate solution into the pump chamber during the second stage of the retraction stroke. Thus, the base dialysate solution and the concentrated dialysate solution are mixed directly in the pump chamber 138 of the disposable cassette 112.

At 1302, the PD machine 102 configures the disposable cassette 112 to open a first fluid pathway between a reservoir 920 and a pump chamber 138. The reservoir 920 contains base dialysate solution at a first concentration.

At 1304, the PD machine 102 draws a first volume of base dialysate solution from the reservoir 920 during a first portion of the retraction stroke.

At 1306, the PD machine 102 configures the disposable cassette 112 to open a second fluid pathway between a dialysate bag 930 and the pump chamber 138. The dialysate bag 930 contains concentrated dialysate solution at a second concentration. The first fluid pathway between the reservoir 920 and the pump chamber 138 is closed when the second fluid pathway is opened.

At 1308, the PD machine 102 draws a second volume of concentrated dialysate solution from the dialysate bag 930 during a second portion of the retraction stroke. It will be appreciated that step 1304 and 1308 are performed during a single retraction stroke of the piston 133.

At 1310, the PD machine 102 configures the disposable cassette 112 to open a third fluid pathway between the pump chamber 138 and a patient line 1002. The pump chamber 138 now contains the mixed target dosage dialysate solution at a third concentration.

At 1312, the PD machine 102 transfers a third volume of target dosage dialysate solution from the pump chamber 138 to the patient line 1002 during a compression stroke of the piston 133.

In some embodiments, either method 1200 or method 1300 can be utilized with an intermediate mixing bag 940. Instead of immediately transferring the dialysate solutions directly from the pump chamber 138 of the disposable cassette 112 to the patient line 1002 to be introduced to the patient, the target dosage dialysate solution can first be stored temporarily in a mixing bag 940 allowing for further mixing of the solutions and heating to a desired temperature in the mixing bag 940. In some embodiments, the mixing bag 940 is coupled to a heater that may warm the dosage dialysate solution prior to transferring the heated solution to the patient. This mixing bag 940 can then be used like any normal dialysate bag during treatment.

In some embodiments, the use of the mixing bag 940 may be performed as the principal dialysate mixing operation, alternatively or additionally to performing mixing using pump chambers. This embodiment may be suitable where a pumping system is used that does not utilize pumping chambers, such as with the use of a peristaltic pump, like that described in connection with the PD machine 300. Operation of the peristaltic pump, e.g. positive displacement caused by rotary motion of pump rollers, may be controlled to perform dialysate mixing according to the aspects and techniques described herein.

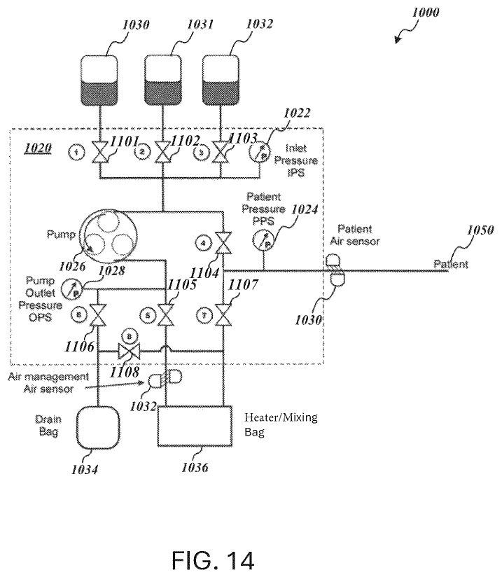

FIG. 14 illustrates a schematic fluid flow diagram for a dialysis system 1000 having a peristaltic pump and utilizing a mixing bag, in accordance with some embodiments. The dialysis system 1000 may include a dialysis machine 1020 operative to facilitate dialysis of a patient 1050. The dialysis machine 1020 may be similar to the PD machine 300 discussed elsewhere herein and include having a peristaltic pump 1026. In some embodiments, the dialysis machine 1020 may include a plurality of valves 1101-1108 operative to manage the movement of fluid within the dialysis machine 1020 and according to techniques like that discussed elsewhere herein. A plurality of supply components 1030-1032 may be arranged to supply dialysate concentrate solutions, e.g. having component features like that of the water purifier 910, the water reservoir 920 and/or one or more bags like that of the dialysate bag 930 as described elsewhere herein, to the dialysis machine 1020. In various embodiments, the dialysis machine 1020 may be associated with various pressure sensors to determine the pressure of fluid within certain portions of the dialysis machine 1020, such as an inlet pressure sensor (IPS) 1022, a patient pressure sensor (PPS) 1024, and/or an outlet pressure sensor (OPS) 1028. In exemplary embodiments, the dialysis machine 1020 may be associated with various air sensors, including a patient air sensor 1030 and/or an air management air sensor 1032. In some embodiments, the pump 1026 may be operative to pump fluid (i.e., purified water, base dialysate solution and/or concentrated dialysate solutions) from the supply components 1030-1032 throughout portions of the dialysis machine 1020 (for instance, depending on an open/close status of the valves 1101-1108). In various embodiments, the dialysis machine 1020 may be associated with a drain bag 1034 and a heater/mixing bag or other container 1036, in which heating and/or mixing of dialysate may be performed as discussed elsewhere herein (e.g., mixing of base dialysate and concentrated dialysate solutions using the mixing bag 940).

Finally, in some embodiments, rather than using a large difference in concentrations between the base dialysate solution and the concentrated dialysate solution (e.g., 0% to 1.5%, and 50%, respectively), two different batches of dialysate solution can be made on-site using two different reservoirs. In a first reservoir, a low concentration dialysate solution is pre-mixed using purified water and the concentrated dialysate solution (e.g., 50% concentration) to get a first low concentration dialysate solution at the lowest concentration to be prescribed (e.g., 1.5% concentration). In a second reservoir, a high concentration dialysate solution is pre-mixed using purified water and the concentrated dialysate solution (e.g., 50% concentration) to get a second high concentration dialysate solution at the highest concentration to be prescribed (e.g., 4.5%). The PD machine 102 or 200 or 300 can then mix a ratio of the high concentration dialysate solution with the low concentration dialysate solution to get a dosage dialysate solution at a target concentration between 1.5% and 4.5%, for example. The mixing mode utilized with this embodiment can be any of the modes discussed above.

The above description provides a context for mixing any desired target concentration of dialysate solution on-site, enabling physicians to prescribe more precise concentrations of dextrose to be used by their patients. The physicians are no longer constrained to use the particular pre-mixed concentrations that are commonly available direct from a manufacturer. In many cases, these techniques can be implemented with legacy PD machines with software updates, when those PD machines include various electronic control components, such as the computer system described below.

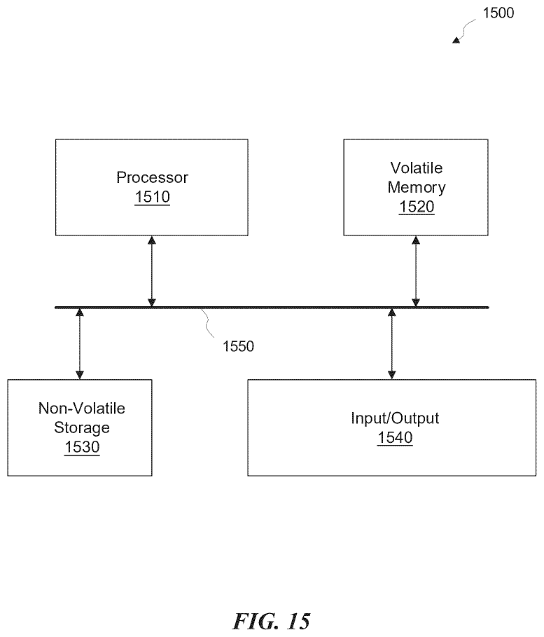

FIG. 15 illustrates an exemplary computer system 1500, in accordance with some embodiments for operating PD machine 102 or 200 or 300. It will be appreciated that, in various embodiments, the control unit 139 can be implemented, at least in part, to include the components of the computer system 1500. The processor 1510 can execute instructions that cause the computer system 1500 to implement the functionality of the control unit 139, as described above.

As depicted in FIG. 15, the system 1500 includes a processor 1510, a volatile memory 1520, a non-volatile storage 1530, and one or more input/output (I/O) devices 1540. Each of the components 1510, 1520, 1530, and 1540 can be interconnected, for example, using a system bus 1550 to enable communications between the components. The processor 1510 is capable of processing instructions for execution within the system 1500. The processor 1510 can be a single-threaded processor, a multi-threaded processor, a vector processor that implements a single-instruction, multiple data (SIMD) architecture, a quantum processor, or the like. The processor 1510 is capable of processing instruction stored in the volatile memory 1520. In some embodiments, the volatile memory 1520 is a dynamic random access memory (DRAM). The instructions can be loaded into the volatile memory 1520 from the non-volatile storage 1530. In some embodiments, the non-volatile storage 1530 can comprise a flash memory such as an EEPROM. In other embodiments, the non-volatile storage 1530 can comprise a hard disk drive (HDD), solid state drive (SSD), or other types of non-volatile media. The processor 1510 is configured to execute the instructions, which cause the PD machine 102 to carry out the various functionality described above.

In some embodiments, the memory 1520 stores information for operation of the PD machine 102 or 200 or 300. For example, the operating parameters can be stored in the memory 1520. The processor 1510 can read the values of the operating parameters from the memory 1520 and then adjust the operation of the PD machine 102 or 200 or 300 accordingly. For example, a speed of the pistons 133A, 133B can be stored in or written to the memory 1520 and read from the memory 1520. The speed is then used to control signals transmitted to the stepper motor drivers. As another example, network parameters for automatically connecting the controller 139 to a WLAN can be stored in the memory 1420.

The I/O device(s) 1540 provides input and/or output interfaces for the system 1500. In some embodiments, the I/O device(s) 1540 include a network interface controller (NIC) that enables the system 1500 to communicate with other devices over a network, such as a local area network (LAN) or a wide area network (WAN) such as the Internet. In some embodiments, the non-volatile storage 1530 can include both local and remote computer readable media. The remote computer readable media can refer to a network storage device such as a storage area network (SAN) or a cloud-based storage service. The I/O device(s) 1540 can also include, but are not limited to, a serial communication device (e.g., RS-232 port, USB host, etc.), a wireless interface device (e.g., a transceiver conforming to WiFi or cellular communication protocols), a sensor interface controller, a video controller (e.g., a graphics card), or the like.

It will be appreciated that the system 1500 is merely one exemplary computer architecture and that the control unit 139 or other processing devices can include various modifications such as additional components in lieu of or in addition to the components shown in FIG. 15. For example, in some embodiments, the control unit 139 can be implemented as a system-on-chip (SoC) that includes a primary integrated circuit die containing one or more CPU core, one or more GPU cores, a memory management unit, analog domain logic and the like coupled to a volatile memory such as one or more SDRAM integrated circuit dies stacked on top of the primary integrated circuit dies and connected via wire bonds, micro ball arrays, and the like in a single package (e.g., chip). The chip can be included in a chipset that includes additional chips providing the I/O device 1540 functionality when connected to the SoC via a printed circuit board.

The system and techniques described herein are discussed for illustrative purposes principally in connection with a particular type of PD cycler, for example a PD cycler having piston-based pumps and a heater tray used to batch heat dialysate in a heater bag, but it is noted that the system and techniques described herein may be suitably used in connection with other types and configurations of dialysis machines and/or medical devices involving the transmission of fluid to and from a patient via a patient line. For example, the system and techniques described herein may be used in connection with a PD cycler using a different configuration and style of pump, such as a peristaltic pump, and may be used in connection with other types of dialysate heating arrangements, such as in-line heating arrangements. Further, the system described herein may be suitably used in connection with other types of dialysis machines, including, for example, hemodialysis machines, and with other types of medical equipment that is unrelated to dialysis treatment, in which on-site mixing of variable medical solution concentrations is desirable.

It is noted that the techniques described herein may be embodied in executable instructions stored in a computer readable medium for use by or in connection with a processor-based instruction execution machine, system, apparatus, or device. It will be appreciated by those skilled in the art that, for some embodiments, various types of computer-readable media can be included for storing data. As used herein, a “computer-readable medium” includes one or more of any suitable media for storing the executable instructions of a computer program such that the instruction execution machine, system, apparatus, or device may read (or fetch) the instructions from the computer-readable medium and execute the instructions for carrying out the described embodiments. Suitable storage formats include one or more of an electronic, magnetic, optical, and electromagnetic format. A non-exhaustive list of conventional exemplary computer-readable medium includes: a portable computer diskette; a random-access memory (RAM); a read-only memory (ROM); an erasable programmable read only memory (EPROM); a flash memory device; and optical storage devices, including a portable compact disc (CD), a portable digital video disc (DVD), and the like.

It should be understood that the arrangement of components illustrated in the attached Figures are for illustrative purposes and that other arrangements are possible. For example, one or more of the elements described herein may be realized, in whole or in part, as an electronic hardware component. Other elements may be implemented in software, hardware, or a combination of software and hardware. Moreover, some or all of these other elements may be combined, some may be omitted altogether, and additional components may be added while still achieving the functionality described herein. Thus, the subject matter described herein may be embodied in many different variations, and all such variations are contemplated to be within the scope of the claims.

To facilitate an understanding of the subject matter described herein, many aspects are described in terms of sequences of actions. It will be recognized by those skilled in the art that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar references in the context of describing the subject matter (particularly in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. Furthermore, the foregoing description is for the purpose of illustration only, and not for the purpose of limitation, as the scope of protection sought is defined by the claims as set forth hereinafter together with any equivalents thereof. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

What is claimed is:

1. A system for peritoneal dialysis (PD) treatment, the system comprising:
a water purifier coupled to a water source;
a reservoir configured to hold base dialysate solution at a first concentration, wherein the base dialysate solution includes a mixture of purified water from the water purifier and at least one of electrolytes or dextrose; and
a PD machine configured to accept a disposable cassette, wherein the PD machine is configured to:
mix a first volume of the base dialysate solution from the reservoir with a second volume of concentrated dialysate solution at a second concentration to create a target dosage dialysate solution at a third concentration between the first concentration and the second concentration.

2. The system of claim 1, wherein the water purifier comprises at least one of a mechanical filter, an activated carbon filter, a reverse osmosis membrane, or a deionization filter.

3. The system of claim 2, wherein the water purifier further comprises a sterilization component that comprises at least one of a ultraviolet light source or an endotoxin filter.

4. The system of claim 1, wherein the first concentration of the base dialysate solution is between 0.0% and 1.5% by weight of dextrose.

5. The system of claim 1, wherein the first concentration of the base dialysate solution is 0.0% by weight of dextrose, and the base dialysate solution contains at least some concentration of electrolytes.

6. The system of claim 1, wherein the disposable cassette is configured to be connected to a first fluid line connected to the reservoir, and wherein the disposable cassette is configured to be connected to a second fluid line connected to a dialysate bag containing the concentrated dialysate solution.

7. The system of claim 6, wherein the PD machine is configured to:
configure the disposable cassette to create a first fluid path between the first fluid line and a first pump chamber;
draw the first volume of the base dialysate solution from the reservoir into the first pump chamber;
configure the disposable cassette to create a second fluid path between the second fluid line and the first pump chamber;
draw the second volume of the concentrated dialysate solution from the dialysate bag containing the concentrated dialysate solution into the first pump chamber;
configure the disposable cassette to create a third fluid path between the first pump chamber and a patient line; and
pump the first volume of the base dialysate solution and the second volume of the concentrated dialysate solution into the patient line.

8. The system of claim 7, wherein:
the PD machine is configured to mix the first volume of the base dialysate solution and the second volume of the concentrated dialysate solution in the first pump chamber; or
the PD machine is configured to mix the first volume of the base dialysate solution and the second volume of the concentrated dialysate solution, at least partially, in the patient line; or
the PD machine is configured to mix the first volume of the base dialysate solution and the second volume of the concentrated dialysate solution in a mixing bag fluidly coupled with the disposable cassette via a third fluid line.

9. The system of claim 8, wherein the PD machine is configured to use partial strokes of a piston to draw at least one of the first volume of the base dialysate solution or the second volume of the concentrated dialysate solution into the pump chamber.

10. The system of claim 1, wherein the PD machine is further configured to pump a volume of the dosage dialysate solution into a patient line attached to the disposable cassette, wherein the patient line is connected to a catheter inserted into a peritoneal cavity of a patient.

11. A peritoneal dialysis (PD) machine, comprising:
at least one pump mechanism proximate an interface for a disposable cassette, wherein the disposable cassette includes at least one pump chamber that interfaces with the at least one pump mechanism; and
at least one processor coupled to a memory, the memory storing instructions that, when executed by the at least one processor, cause the PD machine to:
mix a first volume of a base dialysate solution at a first concentration from a reservoir with a second volume of concentrated dialysate solution at a second concentration to create a target dosage dialysate solution at a third concentration between the first concentration and the second concentration.

12. The PD machine of claim 11, wherein the first concentration of the base dialysate solution is between 0.0% and 1.5% by weight of dextrose.

13. The PD machine of claim 11, wherein the first concentration of the base dialysate solution is 0.0% by weight of dextrose, and the base dialysate solution contains at least some concentration of electrolytes.

14. The PD machine of claim 11, wherein the disposable cassette is connected to a first fluid line connected to the reservoir, and wherein the disposable cassette is connected to a second fluid line connected to a dialysate bag containing the concentrated dialysate solution.

15. The PD machine of claim 14, wherein the PD machine is configured to:
configure the disposable cassette to create a first fluid path between the first fluid line and a first pump chamber;
draw the first volume of the base dialysate solution from the reservoir into the first pump chamber;
configure the disposable cassette to create a second fluid path between the second fluid line and the first pump chamber;
draw the second volume of the concentrated dialysate solution from the dialysate bag containing the concentrated dialysate solution into the first pump chamber;
configure the disposable cassette to create a third fluid path between the first pump chamber and a patient line; and
pump the first volume of the base dialysate solution and the second volume of the concentrated dialysate solution into the patient line.

16. The PD machine of claim 15, wherein:
the PD machine is configured to mix the first volume of the base dialysate solution and the second volume of the concentrated dialysate solution in the first pump chamber; or
the PD machine is configured to mix the first volume of the base dialysate solution and the second volume of the concentrated dialysate solution, at least partially, in the patient line; or
the PD machine is configured to mix the first volume of the base dialysate solution and the second volume of the concentrated dialysate solution in a mixing bag fluidly coupled with the disposable cassette via a third fluid line.

17. A method for operating a peritoneal dialysis (PD) machine, the method comprising:
purifying water using a purifier to generate purified water;
mixing at least one of electrolytes or dextrose with the purified water in a reservoir to create a base dialysate solution at a first concentration;
mixing, using a disposable cassette of the PD machine, a first volume of the base dialysate solution from the reservoir with a second volume of concentrated dialysate solution at a second concentration to create a target dosage dialysate solution at a third concentration between the first concentration and the second concentration.

18. The method of claim 17, wherein the disposable cassette is connected to a first fluid line connected to the reservoir, and wherein the disposable cassette is connected to a second fluid line connected to a dialysate bag containing the concentrated dialysate solution.

19. The method of claim 18, wherein the mixing the first volume of the base dialysate solution with the second volume of concentrated dialysate solution comprises:
configuring the disposable cassette to create a first fluid path between the first fluid line and a first pump chamber;
drawing the first volume of the base dialysate solution from the reservoir into the first pump chamber;
configuring the disposable cassette to create a second fluid path between the second fluid line and the first pump chamber;
drawing the second volume of the concentrated dialysate solution from the dialysate bag containing the concentrated dialysate solution into the first pump chamber;
configuring the disposable cassette to create a third fluid path between the first pump chamber and a patient line; and
pumping the first volume of the base dialysate solution and the second volume of the concentrated dialysate solution into the patient line.

20. The method of claim 17, wherein:
the PD machine is configured to mix the first volume of the base dialysate solution and the second volume of the concentrated dialysate solution in the first pump chamber; or
the PD machine is configured to mix the first volume of the base dialysate solution and the second volume of the concentrated dialysate solution, at least partially, in the patient line; or
the PD machine is configured to mix the first volume of the base dialysate solution and the second volume of the concentrated dialysate solution in a mixing bag fluidly coupled with the disposable cassette via a third fluid line.
